(19) United States
(12) Patent Application Publication
(10) Pub. No.: US 2023/0398261 A1
(43) Pub. Date: Dec. 14, 2023

(54) Process for seamless connecting/joining of tissue comprising crosslinkable groups

(71) Applicant: BIOTRONIK AG, Buelach (CH)

(72) Inventors: Alexander Rzany, Nuernberg (DE); Daniel Niopek; Nina Foh, Forchheim (DE); Bernhard Hensel, Erlangen (DE)

(21) Appl. No.: 18/250,281

(22) PCT Filed: Oct. 28, 2021
(86) PCT No.: PCT/EP2021/080037

§ 371 Date: Apr. 24, 2023

(30) Foreign Application Priority Data
Oct. 30, 2020 (EP) 20205062.1

(51) Int. Cl. A61L 27/36; A61L 27/24; A61L 31/00

(52) U.S. Cl. CPC A61L 27/3691; A61L 27/24; A61L 31/005

(57) ABSTRACT

A process for chemical crosslinking of tissue joining partners including crosslinkable groups, such as free amino groups, by a suitable crosslinking agent under static, quasi-static or periodic pulsatile pressure compression. The compression can be in a defined overlap region for seamless, dense and tight tissue closure. This results in a seamless, homogeneous, and at the same time mechanically stable connection/joining of tissue/tissue components. Seamless connected tissue is provided that includes a piece of tissue having at least two tissue parts overlapping each other and the at least two tissue parts overlapping each other that are materially bonded to each other via crosslinked groups of the tissue.

### PRIORITY CLAIM

This application is a 35 U.S.C. 371 US National Phase and claims priority under 35 U.S.C. § 119, 35 U.S.C. 365(b) and all applicable statutes and treaties from prior PCT Application PCT/EP2021/080037, which was filed Oct. 28, 2021, which application claimed priority from European Application Serial Number 20205062.1, which was filed Oct. 30, 2020.

### FIELD OF THE INVENTION

The invention concerns cross-linking of substantially non-crosslinked tissue, including biological tissue. An example application of the invention is to make Transcatheter aortic valve implantation (“TAVI”), or transcatheter aortic valve replacement (“TAVR”), or percutaneous aortic valve replacement (“PAVR”) implants.

### BACKGROUND

Transcatheter aortic valve implantation (“TAVI”), or transcatheter aortic valve replacement (“TAVR”), or percutaneous aortic valve replacement (“PAVR”) is a minimally invasive procedure in which an artificial aortic valve prosthesis is placed and released in a collapsed (crimped; compressed) state within the native aortic valve. The implant usually consists of individual, manually sutured, collagen-containing tissue components integrated into a suitable self-expanding or mechanically expandable stent (e.g., balloon-expandable) or support structure. Through the typically complex and error-prone suturing process, a complex, three-dimensional tissue geometry is thereby created, which is essential for the functionality of the prosthesis. At the same time, a skilled person is aware that the numerous surgical nodes/sutures represent mechanical weak points that can potentially lead to failure of the implant, and thus can also sometimes cause severe complications in the patient.

There are basically three different types of prosthetic heart valves, especially aortic valve prostheses: Prostheses with mechanical valves, which are manufactured artificially, mostly from graphite coated with pyrolytic carbon; prostheses with valves made from biological tissue (or partly biological tissue locally reinforced by artificial fibers, if necessary), mostly pericardial tissue typically derived from animal sources (e.g., porcine or bovine); and valves made from artificial materials such as polymers. The heart valve formed from the biological tissue is generally secured in a base body (e.g., a solid plastic scaffold or a self-expanding stent or a balloon-expanding stent) and this is implanted in the position of the natural valve. The present invention describes, among other things, a method for sutureless and integral connection/joining of such a tissue for use in a prosthetic aortic valve to be implanted in place of a natural aortic valve.

Usually, the initial tissue must be thoroughly cleaned and prepared before implantation. As far as possible, the tissue is modified in such a way that it is not recognized by the body as foreign tissue, calcifies as little as possible and has the longest possible service life. Essentially, such a process for preparing tissue includes several steps: One possible preparation step is the so-called decellularization of the tissue. In this step, cell membranes, intracellular proteins, cell nuclei and other cellular components are almost completely removed from the tissue to obtain an (approximately pure) extracellular matrix. Cells and cellular components remaining in the tissue represent in particular a possible cause of undesired calcification of the biological implant material. Decellularization should be carried out so gently that the structure of the extracellular matrix and in particular the collagen fibers in the extracellular matrix remain as unaffected as possible, while on the other hand all cells and cellular components contained therein are removed from the tissue as completely as possible.

After decellularization, as many cellular components as possible are removed from the tissue and the biological material consists exclusively of extracellular matrix. In pericardial tissue, the extracellular matrix is predominantly formed from the said collagen fibers. In order to achieve a biological material with the best possible mechanical properties and to prevent defense reactions of the receiving body, in the prior art the collagen fibers are crosslinked by a suitable crosslinking agent through the incorporation of chemical bonds.

The crosslinking agent specifically binds to free amino groups of the collagen fibers and forms chemically stable bonds between the collagen fibers. In this way, a long-term stable biological material is formed from the three-dimensionally arranged collagen fibers, which, moreover, is no longer recognized as foreign biological material. The three-dimensional crosslinking or linking of the individual collagen fibers via the crosslinking agent significantly increases the stability and stressability of the tissue. This is particularly crucial when used as the tissue of a heart valve, where the tissue must open and close as a valve every second.

According to the prior art, the tissue treated in this way is attached to a basic body (e.g., a hollow cylindrical nitinol stent), far predominantly by suturing using a plurality of surgical knots. The main body or scaffold is implantable by surgical techniques (mostly catheter-based). Frequently, the basic scaffold is self-expanding or mechanically expandable with the aid of a balloon, so that the prosthetic heart valve can be guided to the implantation site in a compressed state by a catheter and implanted within the natural valve. In the prior art, such catheter-implantable prosthetic heart valves are usually stored in a storage solution, correspondingly in a moist state. The storage solution serves to sterilely stabilize the biological tissue. One conceivable storage solution is, for example, glutaraldehyde.

For implantation, the prosthetic heart valve must then be removed from the storage solution in the operating room and mounted on the catheter after several rinsing procedures. This assembly of the prosthetic heart valve only in the operating room is cumbersome and labor-intensive. In addition, the correct performance of the assembly depends on the skills of the particular surgical team.

In the case of various medical implants, the problem arises that after implantation, there is a leakage between the surface of the implant and an anatomical structure of the patient, for example, a vessel wall in which the implant was implanted. In the case of a prosthetic heart valve as a medical implant, for example, paravalvular leakage (PVL) may occur, limiting the performance of the prosthetic heart valve.

For example, a method of manufacturing a prosthetic heart valve that includes processing dried biological material has been disclosed in U.S. Pat. No. 8,105,375. According to the method disclosed therein, the biological tissue is fixed or crosslinked with an aldehyde-containing solution (e.g., glutaraldehyde or formaldehyde solution), and treated with at least one aqueous solution containing at least one biocompatible and non-volatile stabilizer prior to drying. Stabilizers include hydrophilic hydrocarbons with a plurality of hydroxyl groups, and examples include water-soluble sugar alcohols such as glycerol, or ethylene glycol or polyethylene glycol.

Basically, heart valve defects (Latin: vitia, singular: vitium) as medical indications for a prosthetic heart valve can be divided into stenoses and insufficiencies according to their functional disturbance. Of all valve vitias, calcifying aortic valve stenosis is the most common acquired valvular heart disease in Western industrialized nations and thus the most common medical indication for heart valve replacement (TAVI/TAVR/PAVR).

A conventionally manufactured transcatheter aortic valve prosthesis typically consists of up to six individual tissue parts/components, which are manually sutured together in a usually extremely time-consuming and cost-intensive process, and then integrated into a stent or other frame structure. This gives the implant a complex, three-dimensional geometry that is essential for the functionality of the prosthesis. The mostly three freely supported, inwardly directed leaflets form semilunar pockets that passively effect valve closure. The additional skirt components (inner and/or outer skirt) attached to the stent/frame structure serve to prevent or seal against paravalvular leakage (PVL). Thus, the tissue portion of a TAVI/TAVR valve usually consists of a total of six individual tissue components cut from crosslinked tissue patches. The three leaflet parts, which functionally effect the opening and closing of the prosthesis, are called “leaflets”. The three so-called inner skirt parts are immovably attached internally to the stent/frame structure in the final product and serve primarily to reduce paravalvular leakage. A shaping process, e.g. laser cutting or punching, is followed by a complex, multi-stage sewing process, which gives the valve implant its characteristic three-dimensional geometry. In some prior art variants, an outer skirt is additionally attached to the outside of the TAVI/TAVR valve, which is also mostly made of tissue and addresses PVL.

The entire valve suturing process is performed entirely manually under a microscope, making it extremely time-, cost-, and resource-intensive. In total, several hundred individual surgical knots are tied, with approximately half of the knots involved in suturing together the aforementioned tissue parts/components and the other half involved in suturing the tissue components into the stent/frame structure. The difficulty here is that if a single knot is placed incorrectly, this immediately leads to rejection of the valve prosthesis and additional costs in the manufacturing process. Furthermore, sutures form mechanical weak points that can potentially lead to failure of the implant—as mentioned at the beginning.

Typically, the manufacturing of a TAVI/TAVR valve starts with the mechanical processing of the tissue (e.g. pericardium), where the required tissue component(s) is/are prepared and cleaned (e.g. from the pericardium). In the subsequent crosslinking process, the tissue is usually placed and/or fixed (e.g., stretched at the edges) on a suitable planar mold (e.g., one or more plates or a plastic frame), and placed in a suitable crosslinking solution (e.g., glutaraldehyde solution including glutaraldehyde oligomers) for several days.

Chemical crosslinking by glutaraldehyde oligomers leads to inter- and intramolecular crosslinking in the collagen, and this is essential to protect the tissue from enzymatic degradation and thus ensure the long-term stability of the implant. In addition, this step forces the tissue into a planar shape, facilitating the laser cutting or a punch-out that typically follows. In this regard, it should be mentioned in general, and without attachment to this theory, that crosslinking in solutions including glutaraldehyde oligomers typically occurs via a plurality of glutaraldehyde macromolecules present in the solution. Due to the large number of molecular variants present, good crosslinking takes place. The spacing of the binding sites on the collagen fibers involved can therefore vary and yet chemically covalent binding can occur due to the glutaraldehyde oligomers.

The background to the need for chemical crosslinking is that biological tissue, unless it is supplied by cells and endogenous processes in the body, is subject to natural decomposition and denaturation processes. Accordingly, it must be specifically processed for further processing into a functional long-term implant. Glutaraldehyde, more correctly called glutardialdehyde, was first used for chemical fixation in the early 1960s and has since become the gold standard for crosslinking collagen-containing tissues. Chemical crosslinking of the collagen structure by glutaraldehyde reduces the immune response and prevents enzymatic degradation after implantation—without compromising the anatomical integrity of the tissue and the viscoelastic properties of the collagen. In addition to its crosslinking property, it can also be used as a sterilizing agent, as it has a killing effect against bacteria, viruses and spores. The great success of glutaraldehyde is due to its commercial availability at low cost, as well as its excellent solubility and high reactivity.

As exemplified above for TAVI/TAVR valves, artificial compounds of tissues/components (biological and/or artificial), especially tissues for medical use, are known. However, the connections of the prior art to that effect are far predominantly made of surgical materials; in particular, surgical sutures including one or more surgical knots. As mentioned, such surgical sutures usually have to be placed manually. This process is very time-consuming, expensive and error-prone—to list just a few of the associated disadvantages. Surgical knots, for example, must be placed individually by personnel in a highly concentrated manner and must always be visually inspected. In addition, each individual knot represents a potential weak point of the medical tissue, since mechanical forces occurring under stress of a medical implant are focused on the knots. Surgical sutures also have a non-negligible space requirement (space requirement), which means that minimum structural sizes of a few millimeters cannot be undercut, especially in the case of medical implants. This noticeably limits medical implants in their medical application areas.

### SUMMARY OF THE INVENTION

A process for chemical crosslinking of tissue joining partners including crosslinkable groups, such as free amino groups, by a suitable crosslinking agent under static, quasi-static or periodic pulsatile pressure compression. The compression can be in a defined overlap region for seamless, dense and tight tissue closure. This results in a seamless, homogeneous, and at the same time mechanically stable connection/joining of tissue/tissue components. Seamless connected tissue is provided that includes a piece of tissue having at least two tissue parts overlapping each other and the at least two tissue parts overlapping each other that are materially bonded to each other via crosslinked groups of the tissue.

### BRIEF DESCRIPTION OF THE DRAWINGS

Embodiments of the invention and further features and advantages of the invention will be described hereafter based on the figures. In the drawings:

FIG. 1 shows a tissue placement of planar tissue patches/components porcine pericardium for a subsequent seamless joining/connection of two tissue patches porcine pericardium according to a process according to the invention;

FIG. 2 shows a planar seamlessly connected/joined porcine pericardium formed by a process according to the invention;

FIG. 3 shows an exemplary three-dimensional initial shape for the subsequent quasi-static or periodic pulsatile pressure loading/compression of porcine pericardial tissue components in the presence of a suitable crosslinking agent according to a process of the invention;

FIG. 4 shows a one-piece, seamlessly connected/joined one-piece valve component formed according to a process of the invention;

FIG. 5 shows an exemplary construction of an inflatable sleeve for the sutureless joining/connection of a TAVI/TAVR valve according to a process of the invention;

FIG. 6 shows an exemplary TAVI/TAVR valve with a self-expanding nitinol stent having a seamlessly joined valve component according to the invention;

FIG. 7 shows an exemplary structure of a device for applying static, quasi-static, or periodic pulsatile pressure to an overlap region of a tissue/component to be joined according to the invention;

FIG. 8A shows an exemplary support surface for tissue to be joined/connected with two holes for fixing and stacking the support surface(s) to implement a process of the invention;

FIG. 8B shows a receiving unit for one or more of the support surfaces according to FIG. 8A;

FIG. 8C shows a stamping device which fits exactly to the support of FIG. 8A;

FIG. 9 shows a three-dimensional molded body with a support surface seamless joining/connection of a one-piece valve component of a TAVI/TAVR valve for a process according to the invention;

FIG. 10 shows an assembled variant of the molded body described previously in FIG. 9;

FIG. 11 shows a fully assembled variant of multiple stacked support surfaces with tissues to be joined/crosslinked in the receiving unit of FIG. 8B and a stamping device pressurized according to FIG. 8C;

FIG. 12 shows a negative of a component of the molded body according to FIG. 9 from a rear left perspective;

FIG. 13 shows an assembled variant of the molded body of FIG. 9 for clamping in a crosslinking unit via additionally added edge elements;

FIG. 14 shows a negative of a component of the molded body of FIG. 9 from a front right perspective;

FIG. 15 shows another variant of a pressure die which can be used, for example, to realize a seamless tissue connection of a one-piece TAVI/TAVR valve according to a process of the invention;

FIG. 16 shows an exemplary holder of a crosslinking unit for seamless joining of a one-piece tissue component of a TAVI/TAVR valve according to a process of the invention;

FIG. 17 shows a molded body clamped for a TAVI/TAVR valve in the holder of FIG. 16; and

FIG. 18 shows the device according to FIG. 17, but with pressure-loaded stamp element on the support surface of FIG. 17.

### DETAILED DESCRIPTION OF THE PREFERRED EMBODIMENTS

The present invention relates to a process for seamlessly joining biological and/or artificial tissue including crosslinkable groups, e.g. free amino groups, for use as a component of a medical implant according to the generic term of claim 1, in particular for use as a component of a covered stent or an artificial heart valve, as well as a medical implant according to claim 11, which contains such seamlessly joined biological and/or artificial tissue. More specifically, the present invention relates to a process for seamlessly and materially bonded joining/connecting tissues suitable for chemical crosslinking; for example, elastin-containing tissues and tissues containing free amino groups—in particular, collagen-containing tissues. Preferred in the context of the invention are amino group-containing, more preferably collagen-containing, biological and/or artificial tissue components, such as e.g. biological pericardial tissue components (skirt/leaflets etc.) of a TAVI/TAVR valve or preferred are amino group-containing, more preferably collagen-containing, biological and/or artificial tissue components of a so-called covered stent.

The invention is described herein essentially using the example of a method for the sutureless and material bonded connection/joining of tissue for use for an artificial aortic valve (TAVI/TAVR). While the invention is particularly well suited for joining such tissue, it is not limited to such application(s). For example, the present invention is also applicable to the sutureless and material bonded ex vivo connection/joining of (artificial) blood vessels, (artificial) bone cartilage, (artificial) ligaments, (artificial) skin or the like.

Preferably, according to the invention, the biological and/or artificial tissue is subjected to a pretreatment including an optional decellularization with a suitable detergent, preferably with a solution containing surfactin and deoxycholic acid. The decellularization can also be performed otherwise, for example, via lysis of the cells or by an osmotic digestion.

The tissue according to the invention is to be understood as biological tissue. Biological tissue preferably has an organizational level intermediate between cells and a complete organ. The biological tissue may be an autologous, xenogeneic or allogeneic tissue. In principle, all types of tissue e.g. from non-mammalian or mammalian tissue including human tissue can be used. The tissue may be derived from pig (porcine tissue), sheep, goat, horse, crocodile, kangaroo, ostrich, monkey, preferably primate, octopus, rabbit or cattle (bovine tissue). Tissue that can be used may be pericardial tissue, skin, ligament, connective tissue, tendons, peritoneal tissue, dura mater, tela submucosa, in particular of the gastrointestinal tract, or pleura. The tissue can be in its native form or in a processed form or can include combinations thereof.

Autologous tissue (in medicine) refers to tissue that was isolated from the human or animal body and is to be re-transplanted elsewhere in the same human or animal body (i.e. originating from the same human or animal body or in other words donor and recipient are the same). The autologous tissue can be in its native form or in a processed form or can include combinations thereof. The autologous tissue to be used includes chemically and/or biochemically crosslinkable groups.

Allogeneic tissue (in medicine) refers either to material that was isolated from a(nother) human or animal body that is genetically distinct from the human or animal body, but of the same species. Thus, allogeneic (also denoted as allogenic or allogenous) tissue is tissue that was isolated from a human or animal body which is different from the human or animal body where the implant is to be implanted. Allogeneic tissue can be not from the patient itself (but from a genetic different donor of the same species). Allogeneic here also includes hemiallogeneic (genetically different because of being derived from one parent of the same species and one parent from another species). The allogeneic tissue can be in its native form or in a processed form or can include combinations thereof. The allogenic tissue to be used includes chemically and/or biochemically crosslinkable groups.

Xenogeneic tissue (in medicine) refers to tissue that was isolated from a human or animal body of a different (heterologous) species. Thus, xenogeneic (also known as xenogenous or xenogenic) tissue is material that was isolated form a human or animal body which is different from the human or animal body where the implant is to be implanted. Xenogeneic tissue may also refer to tissue based on human or animal donor cells (cells obtained from a or the human or animal donor) being cultivated in a bioreactor or being obtained via 3D printing. The xenogeneic material, e.g. tissue, can be in its native form, in a fixed form, in a processed form or can include combinations thereof.

In the context of the invention, the expressions/terms “biological and/or artificial tissue” or similar terminology describe the tissue genera suitable for the processes of the invention for seamless joining/connecting. That is, for example, (purely) biological tissue is tissue of (purely) natural origin, e.g., porcine pericardium taken from a porcine pericardium. (Purely) artificial tissue is tissue that has been artificially produced, for example, from one or more different polymer(s) —e.g., by suitable 3D printing processes or the like. Biological and artificial tissue refers to mixed forms of e.g. a biological basic substance such as porcine pericardium, but including artificial materials, e.g. for local reinforcement of certain tissue regions, which are exposed to e.g. enormous physiological pressure and/or tensile loads—e.g. leaflets of a TAVI/TAVR valve. However, in the context of the invention, common to all these tissue types, and essential, is that they include crosslinkable groups, e.g. free amino groups (also denoted as —NH₂ group), in particular collagen fibers, which are (chemically and/or biochemically) crosslinkable.

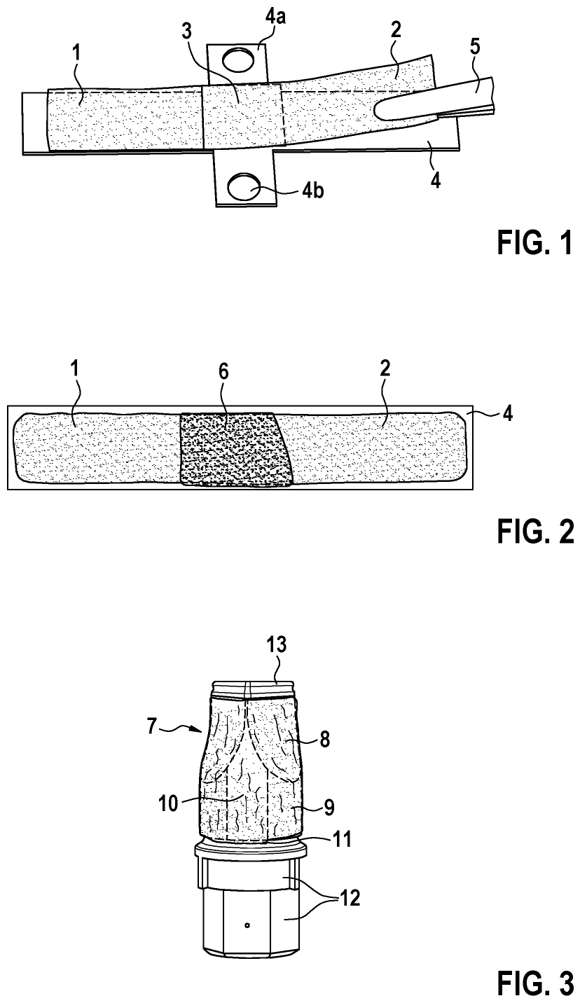

It is also essential for the processes according to the invention that the starting tissue/components which are introduced into the processes according to the invention are substantially non-crosslinked at least in the overlap region (i.e. the tissue region(s) to be joined/connected; see, for example, (3) in FIGS. 1 and (6) in FIG. 2), but preferably in its entirety; i.e. that, if possible, no substantial pre-crosslinking has taken place, for example by glutaraldehyde solution. Substantially non-crosslinked with respect to tissue throughout the application means that the tissue includes (chemically and/or biochemically) crosslinkable groups, preferably more than 50% (chemically and/or biochemically) crosslinkable groups. This means that the proportion of crosslinkable groups in the tissue to be treated is greater than 50%, preferably greater than 60%, even more preferably greater than 80%, most preferably greater than 90%. However, this also means that lightly or only slightly pre-crosslinked or partially crosslinked tissue is suitable for the processes of the present invention.

The processes according to the present invention are thus suitable for seamless joining/connecting of substantially non-crosslinked tissue, native tissue, non-crosslinked decellularized tissue or non-crosslinked non-decellularized tissue. Also suitable are natively dried tissues, which optionally have also been previously subjected to decellularization. The prerequisite is always that the tissue to be joined/connected must include crosslinkable groups, e.g. free amino groups, in particular collagen, e.g. contained in collagen fibers.

The invention provides processes that enable, in particular, a seamless and material bonded joining/connecting of tissue/tissue components in a defined area (e.g. one or more overlap area(s)) for its application in medical implants, in particular covered stents and TAVI/TAVR valves.

A chemical crosslinking of tissue joining partners ((1) and (2) of FIGS. 1 and 2) including crosslinkable groups, such as free amino groups, is conducted by a suitable crosslinking agent under static, quasi-static and periodic pulsatile pressure loading, respectively, in a defined overlap region ((3) —FIG. 1 and (6) —FIG. 2) for seamless, dense and firm material closure. As a result, a seamless, homogeneous, and at the same time mechanically stable connection/joining of tissue/tissue components is achieved.

In other words, the invention for the first time specifically exploits, in sufficient quantity and density, the effect that a crosslinking agent such as, for example, glutaraldehyde can also form interfibrillar bonds/crosslinks between two joining partners such as, for example, tissue patches, in order to realize a seamless, tight and stable bond/joint.

The crosslinking agent is preferably an aldehyde-containing crosslinking agent, more preferably glutaraldehyde. In alternative embodiments of the invention, the crosslinking agent includes carbodiimide, formaldehyde, glutaraldehyde acetals, acyl azides, cyanimide, genepin, tannin, pentagalloyl glucose, phytate, proanthocyanidin, reuterin, and/or epoxy compounds.

An exemplary and preferred crosslinking agent is a glutaraldehyde-containing solution consisting of glutaraldehyde at a concentration of 6 g/l in DPBS without calcium and magnesium.

Glutaraldehyde, e.g. in aqueous solution, is a known crosslinking agent, in particular of free amino groups, proteins, enzymes, and e.g. collagen fibers (Isabelle Migneault, Catherine Dartiguenave, Michel J. Bertrand, and Karen C. Waldron: Glutaraldehyde: behavior in aqueous solution, reaction with proteins, and application to enzyme crosslinking; BioTechniques 37:790-802 (November 2004).

A particular advantage of the processes disclosed herein is that, for example, a glutaraldehyde solution can be used as a crosslinking agent in principle independently of concentration.

In one embodiment, for example, the tissue/components to be joined is placed in a glutaraldehyde oligomer-containing solution at pH 7.4 for 48 hours at a temperature of 4° C. during the chemical crosslinking step, and subjected to quasi-static or periodic pulsatile pressure loading/compression.

In general, the skilled person is aware that chemical crosslinking, depending on the tissue to be treated and the desired properties of the crosslinked tissue, can also be regulated or controlled by temperature. Crosslinking generally starts at a temperature above 0° C. Preferred temperature ranges for chemical crosslinking in the sense of the invention are 1-50° C., preferably 10-50° C., more preferably 20-50° C., even more preferably 25-40° C., most preferably 35-40° C., for example at 37° C.

Advantageously, the tissue is rinsed at least once, preferably several times, with a suitable solvent, in particular a buffered salt solution and/or an alcohol solution, before and particularly preferably after the decellularization (provided that it is decellularized tissue). Buffered sodium chloride solutions and/or an ethanol solution are particularly advantageous.

In one embodiment of the present invention, alpha-gal epitopes may additionally be removed from the tissue in a further treatment step, which may be performed after or before the optional decellularization step. Any suitable alpha-galactosidase can be used for such an additional treatment step, e.g., alpha-galactosidase from green coffee bean (GCB) or Cucumis melo.

The invention provides a medical implant having seamlessly and material bonded connected/joined tissue subjected to one of the processes according to the invention.

With the context of the present invention, the term “medical implant” or similar terms particularly includes stent-based implants and heart valve prostheses, particularly aortic valve prostheses, which are stent-based. According to the invention, the term “medical implant” also reads to any medical implant for which the suture-free joined/connected tissue is suitable as a process product, for example, to seal the implant against an anatomical structure.

Also included as a medical implant are pockets that can receive and be implanted with, for example, a cardiac pacemaker, an implantable leadless pacemaker, or a defibrillator.

Nowadays, stents are used particularly frequently as implants for the treatment of stenoses (narrowing of blood vessels). They have a body in the form of a possibly perforated tubular or hollow cylindrical basic structure, which is open at both longitudinal ends. The basic structure of the stent may be composed of individual meshes formed by zigzag or meander-shaped webs. The tubular basic structure of such an endoprosthesis is inserted into the vessel to be treated and serves to support the vessel.

Stents have become particularly popular for the treatment of vascular diseases. The use of stents can widen constricted areas in the vessels, resulting in a gain in lumen. Although the use of stents or other implants can achieve an optimal vessel cross section, which is primarily necessary for the success of the therapy, the permanent presence of such a foreign body initiates a cascade of microbiological processes which, for example, promote inflammation of the treated vessel or necrotic vascular changes and which can lead to a gradual overgrowth of the stent through the formation of plaques.

Stent graft(s)” are stents that contain a fleece or other flat covering, such as a foil or tissue, on or in their often grid-like basic structure. In this context, the term “nonwoven” is understood to mean a textile tissue formed by individual fibers.

In the context of the present invention, the term “nonwoven” also includes the case in which the textile sheet-like structure consists of only a single “continuous” fiber. Such a stent graft is used, for example, to support weak points in arteries, esophagus, or bile ducts, for example in the area of an aneurysm or a rupture of the vessel wall (so-called bail-out device), especially as an emergency stent.

Medical endoprostheses or implants for a wide variety of applications are known in great variety from the prior art and can be combined with the seamless and materially joined tissue of the invention for suitable purposes. Implants in the sense of the present invention are in particular endovascular prostheses or other endoprostheses, e.g. stents (vascular stents, bile duct stents, vascular stents, peripheral stents or, e.g., mitral stents), endoprostheses, endoprostheses or endoprostheses. endoprostheses for closing persistent foramen ovale (PFO), pulmonary valve stents, endoprostheses for closing an ASD (atrial septal defect), as well as prostheses in the area of hard and soft tissue. Also possible as an implant is a left atrial appendage closure device (LAAC).

In an alternative, preferably the medical implant is a prosthetic heart valve, more preferably a TAVI/TAVR valve, including an artificial heart valve made of sutureless and material bonded connected/joined tissue and/or a seal made of said tissue attached, preferably sutured, to an expandable or self-expanding and catheter implantable base frame, stent, or retaining device.

In an alternative, preferably the medical implant is a covered stent or a so-called stent graft, which has one or more tissue components of seamless and material bonded connected/joined tissue and/or a seal of said tissue, which is attached, preferably sutured, to the corresponding basic framework, stent, or holding device, and wherein said covered stent or stent graft is implantable by catheter.

With the context of the invention, the term “covered stent(s)” or similar terms describes an intraluminal endoprosthesis, with a preferably hollow cylindrical basic structure (e.g. made of nitinol), which is covered/sheathed by a further structure and/or one or more material layer(s) on a surface (inside and/or outside), preferably with a seamless and material bonded connected/joined tissue according to the invention. Conceptually, a distinction is to be made in the context of the invention between “covered” in the sense of “covered/jacketed” and “coated” in the sense of “covered with a substance or an alloy”. According to the invention, covered stents refer to stent implants or implants with a retaining structure, wherein the stent or the retaining structure itself is covered or sheathed by the tissue bonded/joined according to the invention, quasi as one or more “layers”. That is, the stent or the retaining structure can, for example, be covered/sheathed from the outside and/or from the inside with the tissue connected/joined according to the invention. This may be realized in the form of one or more layers of the tissue joined/jointed according to the invention; or an inner and an outer layer of this tissue may also be joined/jointed with the joining/joining methods according to the invention, and may also include, for example, an envelope of the tissue according to the invention at one end of the stent/holding structure. For example, an inner layer of the tissue of the invention may be folded over outwardly at both ends of the stent/holding structure, thus becoming an outer layer. The foregoing examples are not limiting, and the person skilled in the art may anticipate several different configurations and possible applications of the tissues joined/jointed according to the invention in light of the present disclosure.

“Coated,” on the other hand, rather focuses on a direct chemical, physical, or pharmaceutical coating of the stent structure. For example, prior art stents can be coated with silicon carbide or a so-called drug eluting stent is coated with a physiologically compatible pharmaceutical agent.

The person skilled in the art is aware here that numerous other conceivable possible applications of the seamlessly and material bonded connected/joined tissue for medical implants according to the invention are to be considered.

In all embodiments of the present invention, the decellularization method, if performed, is applied to tissue that is not conventionally crosslinked after decellularization; rather, crosslinking occurs exclusively in the processes disclosed herein under quasi-static or periodic pulsatile pressure/compression in one or more selected overlap region(s) of the tissues involved.

Such a tissue could be used, for example, in cases where cellular ingrowth is preferred, such as in the treatment of a wound or burn with a porous matrix or when used as a means of sealing an implant or graft.

After the optional decellularization and crosslinking processes disclosed herein, the tissue/tissue component can undergo a dimensional and structural stabilization step. It has also been shown that stabilization of the tissue can be significantly enhanced by exposure to certain stabilizing agents.

In a preferred stabilization step, the tissue is exposed to at least one solution containing glycerol and/or polyethylene glycol, wherein the tissue is exposed to either one of these solutions or to the two solutions sequentially in any order and composition as first and second solutions or to both solutions or even to multiple solutions with different molecular weights of PEG simultaneously as a mixture of solutions or sequentially in any order. When drying tissue, e.g. for storage or transport of the tissue, the stabilization process is preferably carried out before drying.

As a non-limiting example, the stabilization process can be performed, for example, after decellularization and crosslinking by immersing the tissue in a series of one or more stabilizing solutions of glycerol and/or polyethylene glycol to sufficiently saturate the tissue with stabilizing agents and ultimately produce a stable, dry tissue with a seamless joint/joint. Saturation times can vary, but typically take about 5 minutes to 2 hours or 5 minutes to 15 minutes, depending on the properties of the tissue. The stabilized tissue can be dried by placing the tissue, for example, in a suitable environment with constant low relative humidity or, for example, controllable humidity and/or temperature, for example, in a climate chamber or desiccator and reducing the relative humidity. For example, from 95% to 10% over 12 hours at 37° C. The person skilled in the art is aware of the fact that, depending on the circumstances, another suitable drying protocol may be applied.

In general, throughout the present disclosure, the skilled person can suitably adjust the technical parameters such as times, amounts, concentrations, temperatures and, for example, pressures depending on the type of tissue to be treated and the desired crosslinking/bonding results.

The polyethylene glycol-containing solutions typically contain polyethylene glycol with an average molecular weight between 150 g/mol and 6000 g/mol, or a mixture thereof. As used herein, the term “between” includes the upper and lower specified values. Thus, an average molecular weight between 150 g/mol and 6000 g/mol is intended to include 150 g/mol and 6000 g/mol.

In some embodiments, at least one polyethylene glycol-containing solution includes polyethylene glycol having an average molecular weight between 150 g/mol and 200 g/mol, between 150 g/mol and 300 g/mol, between 200 g/mol and 300 g/mol, between 200 g/mol and 600 g/mol, between 200 g/mol and 400 g/mol, between 150 g/mol and 400 g/mol, or between 400 g/mol and 600 g/mol. According to a particularly preferred embodiment, the polyethylene glycol-containing solution provided alone or before or after a glycerol solution contains polyethylene glycol at or about 150 g/mol to 300 g/mol or at or about 200 g/mol (e.g., PEG200), and in an even more preferred embodiment, the polyethylene glycol-containing solution contains 40% PEG200 or about 40% PEG200.

The term “about” as used herein is intended to encompass a variation above and below the stated amount that would be expected in normal use, such as a variation of 5% or 10%.

Glycerin may be added to any of the above stabilizing solutions to form a mixture, or it may be provided separately for stabilizing purposes, such as in aqueous solution.

In some embodiments, a subsequently applied polyethylene glycol-containing solution contains polyethylene glycol having a higher average molecular weight than a previously applied polyethylene glycol-containing solution. In some embodiments, the subsequently applied polyethylene glycol-containing solution contains polyethylene glycol having an average molecular weight between 200 g/mol and 6000 g/mol, or a mixture thereof. In some embodiments, the subsequently applied polyethylene glycol-containing solution includes polyethylene glycol having an average molecular weight between 300 g/mol and 1500 g/mol, or a mixture thereof.

In some embodiments, the subsequently applied polyethylene glycol-containing solution includes polyethylene glycol having an average molecular weight between 400 g/mol and 1200 g/mol, or a mixture thereof. In some embodiments, the subsequently applied polyethylene glycol-containing solution includes polyethylene glycol having an average molecular weight between 400 g/mol and 800 g/mol, or a mixture thereof. In some embodiments, the subsequently applied polyethylene glycol-containing solution includes polyethylene glycol having an average molecular weight between 400 g/mol and 600 g/mol, or a mixture thereof. In some embodiments, the subsequently applied polyethylene glycol-containing solution contains polyethylene glycol having an average molecular weight of 400 g/mol (PEG400) or about 400 g/mol. Again, glycerol may be added to any of the above stabilizing solutions to form a mixture, or it may be provided separately as a stabilizing solution.

In this regard, the skilled person is aware that the temperature during the stabilization step can affect the results. For example, too high a temperature (e.g., above about 85° C.) will cause denaturation and irreversible damage to the tissue crosslinked, e.g., glutaraldehyde crosslinked, for the purpose of bonding/joining. Again, however, too low a temperature can lead to a solution that is too viscous. Preferably, exposure to the stabilizing solutions is at 37° C., but temperatures from room temperature up to 60° C. should be tolerable.

As mentioned at the outset, the processes described in the present invention are suitable for the preparation of substantially non-crosslinked tissue or, for example, decellularized, substantially non-crosslinked tissue—with the proviso that crosslinkable groups, e.g., free amino groups, must be present in the tissue. Optionally, all of the tissues addressed within the scope of the invention may be stabilized as described herein. Optionally, alpha-gal epitopes can be removed from all these tissues by a suitable alpha-galactosidase treatment (preferably originating from GCB or Cucumis melo, see above).

As for the implant itself, the aforementioned problem is further solved by an implant containing biological tissue that has been subjected to one of the processes according to the invention and, if necessary, subsequently stabilized and/or dried.

In this case, the drying of the tissue is designed in such a way that a slow and gentle removal of the water in the liquid state from the tissue is ensured. This is advantageously achieved by the controlled reduction of the ambient humidity of the biological tissue in a suitable environment, such as a desiccator or a climatic chamber, with controlled adjustment of the parameters of the ambient atmosphere of the biological tissue.

The invention includes a surprising realization of the inventors that various suitable crosslinking agents, such as and preferably glutaraldehyde, not only have the ability to form inter- and intramolecular crosslinks within a collagen fiber (see prior art above), but also interfibrillar crosslinks between individual fibers. Thus, it is possible for the first time to generate seamless and materially material bonded connections/joints in an overlapping area of two tissue joining partners, which include crosslinkable groups, such as free amino groups; e.g. containing collagen, by simultaneously applying a quasi-static or periodic pulsatile pressure load/compression by a suitable device. The basic requirement for this is that the distance between the collagen fibers is smaller than the length of the crosslinking molecules involved, such as the glutaraldehyde oligomers mentioned above, which form the actual crosslinks. Therefore, in the context of the invention, a pressure-generating device has been provided to generate a quasi-static or a periodic pulsatile vertical force application (pressure load/compression), with desired repetition cycles and over a desired time period, to a defined tissue region during the crosslinking process. According to the invention, the pressure generating device can be based on the physical principles of pneumatics, mechanics, and, for example, hydraulics, but is not limited in this respect. In the context of the invention, hydraulics is a particularly preferred embodiment for generating the pressure load/compression. The basic requirement for said formation of interfibrillar crosslinks is that the distance between the collagen fibers and microfibrils involved is smaller than the length of the glutaraldehyde oligomers involved (see above), which essentially form the crosslink. Appropriate pressing parameters over suitable time periods to reduce the fiber spacing are thus essential to enable a stable, seamless and material bonded bond using glutaraldehyde oligomers. At the same time, however, a high pressing pressure potentially, and thus not necessarily, results in preventing accessibility of the crosslinking solution to the tissue during force application.

Therefore, according to the invention, in a preferred embodiment, not only a quasi-static pressure on the tissue over a suitable longer period of time during crosslinking is considered (quasi-static refers to a constant pressure over a longer period of time (e.g. 300 seconds), which may be less frequently interrupted by short and suitable pressure pauses (e.g. 1 or 2 second(s)), but a periodic-pulsatile pressure load/compression over suitable shorter periods, but with possibly more frequent repetition of the pressure phases, also interrupted by short pressure pauses (e.g. 30 seconds pressure, 1 or 2 second(s) pressure pause, followed by 30 seconds pressure, 1 or 2 second(s) pause, etc.). That is, in the pressureless phases (pressure pauses) during the crosslinking process, sufficient contact between the crosslinking agent, e.g. the glutaraldehyde oligomers, and the tissue to be joined/connected is ensured in this way. For this purpose, a suitable device is provided with which both a quasi-static, relatively constant pressure can be realized over longer period cycles, and a dynamic, periodic pulsatile pressure can be generated on the tissue, but over shorter and more frequent period cycles.

In an alternative embodiment, it is possible to achieve the crosslinking according to the invention for seamless and material bonded connecting/joining via a static, i.e. permanent pressure, without pauses. The prerequisite for this is to provide a support surface for the tissue to be joined/connected which is perforated, i.e. is continuous for the crosslinking solution, in order to ensure its access to the tissue to be crosslinked.

In some embodiments, the disclosed processes are used to prepare a coronary artery bypass graft. In other embodiments, the processes of the invention are used to prepare biological and/or artificial tissue/tissue components for a heart valve replacement. In other embodiments, said processes are used for seamless and material bonded joining/connecting of tissue, grafts or even substrates for use in a wound treatment process, e.g., for treating lacerations or burns—e.g., wound patches joined/connected according to the invention.

In some embodiments, the processes are used to provide a sutureless and material bonded connection/joint to treat an inguinal hernia. In some embodiments, the processes disclosed herein are used for endogenous tissue regeneration using the patient's body to naturally restore tissue via a biodegradable scaffold. As stated, basic requirements for the foregoing and herein disclosed uses of the processes of the invention are: i) substantially non-crosslinked starting material/starting tissue, and ii) that the substantially non-crosslinked starting material/starting tissue includes crosslinkable groups, e.g., includes free amino groups, and is thus suitable for chemical crosslinking, preferably using glutaraldehyde.

In the context of the present invention, the terms “including amino group(s)”/“including free amino groups” or similar terminology mean that the tissue(s) to be joined/connected must include free amino groups that are chemically crosslinkable by a suitable crosslinking agent in order to be seamlessly and materially bonded joined/connected via the processes described herein.

A preferred embodiment for amino group-containing tissue(s) are collagen-containing tissues such as connective tissue, skin, subcutaneous tissue, ligaments, cartilage, bone, tendons, teeth, and in particular pericardium (porcine and bovine for example), etc. Accordingly, the processes disclosed herein lend themselves particularly to the production of medical implants in the areas of: Skin, wound healing, therapies of burn patients, replacement of ligaments, cartilage, bone, or tendons, and in implantology. It is clear to the skilled person that due to the very broad medical application possibilities of compounds/joints of e.g. collagen-containing biological tissues, the aforementioned listing is by no means to be interpreted as exhaustive.

With this context, the term “collagen-containing(s)” or similar terms used in the context of the invention describes that the tissue(s) to be joined/connected must include free collagen fibers in order to be seamlessly and materially bonded joined/connected via the processes described herein.

Suitable collagen-containing tissues within the scope of the invention are, for example, native collagen-containing tissues, moist collagen-containing tissues, already processed (but substantially non-crosslinked) collagen-containing tissues, such as, for example, already stabilized collagen-containing tissues, already preserved collagen-containing tissues, already dried (non-crosslinked) collagen-containing tissues, already decellularized tissues, as well as mixed forms of the aforementioned tissues. It is clear to the person skilled in the art that this list of suitable collagen-containing tissue forms is not exhaustive, but that further collagen-containing tissue types may be suitable for the disclosed process.

In accordance with the invention, bonding processes for stabilized, dried (non-crosslinked) tissue in particular have been tested.

In an alternative, even the seamless and material bonded connection of already fully or partially crosslinked tissue is possible in principle, whereby, exceptionally, either no crosslinking solution such as, for example, glutaraldehyde solution or only a very low-concentration glutaraldehyde solution (0-1% glutaraldehyde) is additionally required.

This means that a seamless compound in the sense of the invention can not only be formed by the direct, bilateral bonding of free glutaraldehyde oligomers (as described above), but in principle also by polymerization of oligomers already bonded on one side in the overlap region.

This means that fully or partially pre-crosslinked tissue can also be bonded/joined in pure Dulbecco's phosphate-buffered saline (DPBS) in the sense of the invention. Therefore, it can be assumed that the joining mechanism is indeed not exclusively due to the direct, bilateral bonding of free amino groups between the joining partners (tissue/tissue components), but likewise to the polymerization of unilaterally bonded crosslinking molecules in the overlap region.

In a further preferred embodiment, the processes according to the invention provide medical implants having a base structure, wherein a tissue or tissue component obtained according to one of the processes according to the invention is attached/fixed in and/or on the base structure.

In an additional or alternative embodiment, a tissue or tissue component obtained according to one of the processes according to the invention is attached/fixed in at least one section of the stent implant, preferably at the proximal and/or distal end of the implant.

Thereby, the tissue or tissue component can be connected/joined, for example, over the entire length of the implant or, for example, only at the proximal and/or distal ends of the implant by the process according to the invention, in such a way that there is a seamless and material bonded connection/joint, for example, between an inner and an outer side of the implant through the meshes/cells of the implant. This reduces/prevents entirely the suturing of the tissue/tissue component to the implant; e.g. a stent-graft or a so-called covered stent (see above).

Such stent-based implants described above can be used, for example, as a so-called bail-out stent, neurostent, drug eluting stent, graft on balloon (PEB), PTA (percutaneous transluminal angioplasty), artery replacement or vein replacement.

The basic structure of such an implant can preferably be a metal or a metal alloy, preferably stainless steel, CoCr, a magnesium alloy (implant designed as a stent consisting of a magnesium alloy is also called AMS=absorbable metal stent) and/or nitinol, and/or a polymer from the class of biodegradable polymers, preferably polylactic acids, polycaprolactones and/or mixtures or copolymers thereof, and/or a polymer from the class of biocompatible polymers, preferably UHMWPE and/or PEEK.

In a further embodiment, a metallic base structure/stent implant may additionally be provided with a coating of amorphous silicon carbide (aSiC coating).

In a further preferred variant, the medical implant is a vascular valve prosthesis, in particular a heart valve prosthesis. For example, an aortic valve prosthesis, a tricuspid valve prosthesis, a mitral valve prosthesis and a pulmonary valve prosthesis are suitable examples of a heart valve prosthesis. Typically, such prostheses or implants have a stent-like structure that carries a valve assembly inside it to replace a natural vascular or heart valve. In this regard, the seamless and material bonded connected/joined tissue may be applied to a surface of the prosthetic heart valve (internal and/or external).

In a further variant, the medical implant is a dry-stored and/or dry-delivered complete system, in particular a dry-stored/dry-delivered heart valve prosthesis, in particular an aortic valve prosthesis.

In a further variant, the heart valve prosthesis, in particular aortic valve prosthesis, including one or more of the sutureless and tissue-joined/tissue components, is loaded in a dehydrated state into a so-called catheter delivery system and is delivered in this preloaded state to an operating room.

All variations of the sutureless and tissue bonded/joined tissue/tissue component(s) may be combined in any manner and may be transferred in any combination to the medical implant described herein, and vice versa.

In particular, the present invention discloses processes based on which crosslinking by a suitable crosslinking agent, such as, for example, glutaraldehyde solution including glutaraldehyde oligomers, in combination with a quasi-static or preferably periodic pulsatile pressure load/compression, enables a seamless, material bonded and durable connection/joint between the tissue/components (biological and/or artificial) defined above. The joining techniques disclosed herein can achieve, among other things, sutureless, material bonded and durable medical implants, such as, for example, sutureless covered stents or a sutureless TAVI/TAVR valve (each with respect to the tissue components, such as, for example, skirt and/or leaflet elements). In particular, suture-free skirt tissue components of a TAVI/TAVR valve (inner and/or outer skirt) can lead to an improved seal against paravalvular leakage (PVL).

With the context of the invention, the terms/expressions “quasi-static compressive loading/compression” or similar terms/expressions denote an essentially vertical physical application of force to the tissue to be joined/connected, carried out in such a way that it can be considered exclusively as a sequence of equilibrium states. Thus, the time scale on which a quasi-static process occurs must be much slower than the time period in which equilibrium is reached (the so-called relaxation time). Although a respective state of equilibrium prevails to a large extent at each point in time of the process, it is nevertheless generally an objective of the process to obtain different states or a characteristic curve. This means that the equilibrium state at time t1 (pressure load) may well differ considerably from the equilibrium state at time t2 (pressure relief or pressure pause). The above definition is merely intended to exclude the possibility that dynamic or more dynamic processes, e.g. a periodic pulsatile pressure load/compression, have any appreciable influence on the joining/connecting behavior of the tissue components to be joined/connected.

Specifically, in the context of the invention, this means that the relationship between “with pressure loading” and “pressure relief/pressure pause” during the chemical crosslinking process in the case of “quasi-static”, is more protracted over time for the pressure loading, and with longer periods of time, possibly several times alternating, than in the direct The “periodic-pulsatile” relationship of “pressure load” and “pressure relief/pressure pause” is shorter for the pressure load, which means that the two states “with pressure”/“without pressure” are also shorter over time and, if necessary, are repeated alternately much more often.

Conversely, the terms/expressions “periodic-pulsatile pressure loading/compression” or similar terms/expressions denote that the relationship between “pressure loading” and “pressure relief/pressure pause” during the chemical crosslinking process is more short-lived over time, especially for the pressure loading, and thus the states “with pressure”/“without pressure” and with smaller time spans also alternate noticeably more often, in direct comparison to the “quasi-static” conditions described above.

Specifically, in the context of the invention, the terms/expressions “quasi-static pressure load compression” or similar terms/expressions can be used over a ratio of, for example, 300:1 seconds with respect to “with pressure load” (300 seconds) vs. “pressure release/pressure pause” (for example 1 or 2 second(s)), and thus differ from the terms/expressions “periodic-pulsatile pressure load/compression” or similar terms/expressions in such a way that in the latter case a ratio of e.g. 30:1 seconds exists with respect to “with pressure load” (e.g. 30 seconds) versus “pressure relief/pressure pause” (e.g. 1 or 2 second(s)).

That is, “quasi-static” includes, for example, a single, constant pressure load/compression on the tissue to be joined/connected of 5 minutes (=300 seconds) in the presence of a suitable crosslinker solution with, for example, 1 or 2 second(s) pressure relief/pressure pause. Likewise, however, “quasi-static” also describes those cases in which two or more times of constant pressure load/compression with the pressure releases/pressure pauses as described above act on the tissue to be joined/connected. That is, even corresponding multiple cycles of this rather protracted “quasi-static” form of pressure loading and very short pressure pauses in between falls under these terms.

In contrast, this means, for example, that “periodic-pulsatile” includes at least two, but also several, short pressure loads/compressions on the tissue to be joined/connected of, for example, 30 seconds in the presence of a suitable crosslinker solution, but also always with 1 or 2 second(s) pressure relief/pressure pause. This means that even correspondingly multiple cycles of this rather short “periodic-pulsatile” form of pressure loading with short pressure pauses in between fall under these latter terms.

An artisan will understand that it is not necessary to slavishly adhere to the exact ratios of 300:1 seconds for “pressure load” (300 seconds) versus “pressure relief/pressure pause” (e.g. 1 second) in terms of “quasi-static”, and 30:1 for “pressure load” (e.g. 30 seconds) versus “pressure relief” (e.g. 1 second) in terms of “periodic-pulsatile”. 1 second) in terms of “periodic-pulsatile”, but rather the relations of the mentioned time spans to each other distinguish the variants “quasi-static” from “periodic-pulsatile” during chemical crosslinking, and the exact values may suitably deviate from the above examples. For example, for “quasi-static” the above-described ratios of 250:1 seconds or, for example, 350:1 seconds are also conceivable, and with regard to “periodic-pulsatile”, for example, 15:1 seconds or 30:2 seconds are conceivable.

In the pressureless phases, quasi in the pauses of the external pressure load, a sufficient contact between the chemical crosslinking agent (e.g. glutaraldehyde) and the tissue components in the contact area is ensured in this way.

The above-mentioned alternative case of static crosslinking—without pressure pause—but with perforated/hole counterform for accessibility of the crosslinking solution is appropriately delimited with the above definition of the quasi-static case.

In the context of the invention, quasi-static pressure loading/compression is preferred over static pressure loading/compression, and periodic-pulsatile pressure loading/compression is the most preferred embodiment for the processes disclosed herein.

Another factor of the disclosed joining/connecting processes is the total time period over which the static, quasi-static, or periodic-pulsatile pressure loading/compression acts on the tissue being joined/connected during chemical crosslinking.

A static, quasi-static or periodic pulsatile pressure load/compression over a total time duration of 1 to 3 days is preferred. A total time duration that falls below 4 hours may indeed result in a bond/join of the tissue partners involved; however, this appears too unstable to bring about a permanence of the bond/join. According to the invention, sufficient durability of the seamless and integral joints/junctions of the tissue partners is only given from at least 12 hours, preferably at least 24 hours, more preferably at least 36 hours, more preferably at least 48 hours, even more preferably from 72 hours of the static, quasi-static or periodic pulsatile pressure load/compression under the chemical crosslinking by a suitable crosslinking agent.

The skilled person is generally aware that the above-mentioned times can vary considerably depending on the tissue to be treated and the crosslinking agent to be used. Too short times are likely to lead to insufficient stability, too long times are likely to end in a waste of time, and the skilled person would optimize the parameters (time, temperature, concentrations, etc.) depending on the material.

At this point, the overlap length of the tissue components, the physical compression type (hydraulic, mechanical, etc.), cylinder force and the crosslinking time itself should be highlighted as other significant factors influencing the processes according to the invention. Thus, despite a lower breaking load, a reduction in the overlap length tends to result in a higher bond strength. In order to ensure the accessibility of the crosslinking agent, e.g. the glutaraldehyde solution, to the overlap area, a quasi-static or periodic pulsatile pressure load/compression is indispensable according to the invention.

The cylinder force must be selected appropriately, depending on the compression area, in order to bring about significant (collagen) fiber densification.

With regard to the crosslinking duration, a total period of static, quasi-static or periodic pulsatile compressive loading/compression of three days is particularly preferred.

The crosslinking of overlapping tissue joining partners according to the invention is a valid concept for the seamless and material bonded joining/connecting of tissue, in particular tissue containing collagen. With regard to the application itself, however, the skilled person must always take into account the load limits of the bonded joint in different load cases as well as the effects of the compression process on the properties of the tissue joining partners.

The exemplary process described below, represents an embodiment of the invention, and is particularly, but not exclusively, suitable for native (biological) as well as for stabilized (e.g. dried) and/or decellularized tissue. In general, the disclosed processes are suitable for tissues containing collagen.

Thus, the present invention provides a process for seamless, material bonded, and durable joining/connecting of tissue or a tissue component, preferably substantially non-crosslinked tissue/tissue components, for medical applications, in particular for use as a component of a medical implant, preferably a vascular implant, more preferably an artificial heart valve or a covered stent, wherein the process includes at least the following steps:
(a) providing one or more tissue(s) to be joined, preferably substantially non-crosslinked tissue(s) including crosslinkable groups, in particular free amino groups, and having an overlap region;
(b) providing a suitable container, mold and/or support surface for the tissue/tissue component(s);
(c) providing a device capable of receiving the container, mold and/or support surface of step (b) in a form-fit manner, and further capable of providing controllable static, quasi-static or periodic pulsatile and substantially vertical compressive loading/compression of the overlap region(s) of the tissue/tissue component(s) to be joined of step (a), wherein the pressure load/compression is applied in a range of 0.01-10 N/mm², preferably 0.1-1 Nmm², over a time in the range of 1 second to 15 minutes, preferably with pressure relief/pressure pauses of 1 to 60 seconds, and this over a total period of at least 4 hours to a maximum of 12 days;
(d) Optional cutting of the tissue/tissue component(s) to be joined/connected after step a) by a suitable cutting instrument and/or a suitable cutting device;
(e) Placement/arrangement of the tissue/tissue component(s) after step a) or d) in the container, in the mold and/or on the support surface after steps b) and c) for joining/connecting the overlap area;
(f) chemical crosslinking of the tissue/component(s) after step e) in the device after step c) with addition of a suitable crosslinking agent into the container, mold and/or support surface and subsequent application of a quasi-static or periodic pulsatile compressive load/compression to the overlap area(s);
(g) demolding/removal of the tissue/tissue component(s) bonded/joined after step (f);
(h) Optional (purely) chemical post-crosslinking using a suitable crosslinking agent.

According to the invention, said container, mold, support surface may be a two- and/or three-dimensional mold suitable for chemical crosslinking and static, quasi-static, or periodic pulsatile compressive loading/compression, for example, produced by a known 3D printing process (e.g., tooth-lifting process such as CNC milling). The material of the mold must be suitable to enable the process steps disclosed herein without negatively affecting the integrity of the tissue/component(s) to be joined.

A suitable device for the processes disclosed herein is, for example, a pneumatic cylinder and/or inflation sleeve in combination with at least one control element including electronics configured to control a static, quasi-static and/or periodic pulsatile, time-dependent and substantially vertical pressure/compression movement in the overlap region(s) of the tissue/component(s). Substantially vertical or orthogonal means with a deviation of ±10°.

A preferred device for the processes disclosed herein is, for example, a hydraulic cylinder and/or a hydraulic inflation sleeve in combination with at least one control element including electronics configured to control a static, quasi-static and/or periodic pulsatile, time-dependent and substantially vertical pressure/compression movement in the overlap region(s) of the tissue component(s).

As a suitable cutting method of tissue in the sense of the invention, for example, laser cutting by a suitable laser cutting device such as a CO₂ laser or a femtolaser is suitable; however, this is always in combination with a suitable positioning unit for the tissue/tissue component(s). Waterjet cutting is also conceivable.

A suitable cutting instrument in the sense of the invention is, for example, a pair of scissors, a scalpel, a knife, etc.

With the above context of the invention, the disclosed processes include the following essential influencing parameters on the quality of the seamless, material bonded and durable connection/joint of the tissue/tissue component(s):

Static Compressive Load/Compression.

Appropriate compression loading is essential for the seamless, integral and durable joining/connecting of the tissue/tissue component(s) in the static regime. For the static regime, a time interval in the pressure phase of at least 3 minutes up to at least 15 minutes has proven to be suitable. A static pressure load of at least 3, 4, 5, 6, 7, 8, 9, 10, 11, 12, 13, 14 up to at least 15 minutes is therefore suitable; for example, also including 20, 25 or 30 minutes of constant pressure; depending on the dependence of the starting tissue to be joined/connected.

Pressure Loading/Release Times of Quasi-Static Pressure Loading/Compression

Suitable pressure-change times are essential for seamless, material bonded and consistent joining/connecting of the tissue/tissue component(s). For the quasi-static regime, a time interval in the pressure phase of at least 60 seconds up to 15 minutes has proven to be suitable. A quasi-static pressure load of at least 1, 2, 3, 4, 5, 6, 7, 8, 9, 10, 11, 12, 13, 14 up to 15 minutes is therefore suitable; e.g. 60 seconds of pressure as the lower limit and a maximum of 15 minutes of pressure per cycle as the upper limit; e.g. and particularly preferably 5 minutes.

For the pressure-relieving phase/pressure pause, a time interval of at least 1 second but not more than 10 seconds per cycle has proven suitable in the quasi-static regime; e.g. and preferably 1 to 2 seconds.

Pressure load/pressure release cycling times of the periodic pulsatile pressure load/compression. Appropriate pressure-change times are essential for seamless, integral and consistent joining/connecting of the tissue/tissue component(s). For the periodic pulsatile regime, a time interval in the pressure phase of at least one second up to 1 or 4 minutes has proven to be suitable. Suitable is therefore a pressure load of at least 1, 2, 3, 4, 5, 6, 7, 8, 9, 10, 20, 30, 40, 50 seconds or 1, 2, 3, 4 minutes; e.g. 1 second of pressure as lower limit and a maximum of 4 minutes as upper limit; e.g. and preferably 30 seconds.

For the pressure-relieving phase/pressure pause, a time interval of at least 1 second but not more than 10 seconds per cycle has proven to be suitable in the periodic pulsatile regime; e.g. and preferably 1 to 2 seconds.

Compression Pressure

For both the static, the quasi-static and the periodic pulsatile regime, values in the range of 0.01-10 N/mm², in particular 0.1-1 N/mm², have been found to be advantageous as suitable compression pressures for the seamless, integral and durable joining/connecting of the tissue/tissue component in the overlap region according to the invention. At lower compression pressures, the mechanical stability of the joint decreases. Higher compression pressures are rather ineffective or even ineffective with regard to the bond strength of the joint. It should be noted that compression of the tissue/tissue component to be bonded results in fiber compaction and thus also a reduction in thickness, which leads to increased mechanical stiffness and optical transparency of the tissue.

Crosslinking Time—Total Time for the Process According to the Invention

For both the static, quasi-static and the periodic-pulsatile regimes, a total crosslinking time of in particular at least 4 hours, preferably at least 12 hours to 3 days crosslinking (without optional post-crosslinking) with a suitable crosslinking agent, in particular glutaraldehyde, has been proven according to the invention. In principle, a further increase in the crosslinking time is rather ineffective or even ineffective with regard to the adhesive strength of the seamless joint/joint. However, this does not exclude a post-crosslinking in e.g. glutaraldehyde in the “free-floating” state, which typically lasts at least 5 days; but is no longer relevant for the tissue connection/joining according to the invention, only for the final state of the tissue/component(s) as completely reacted biological material.

Furthermore, in embodiments of pericardial tissue/pericardial tissue components, it has been found that the choice of overlapping pericardial sides (rough or smooth; pericardium fibrosum or lamina parietalis, respectively) has no significant influence on the adhesive strength of the sutureless, material bonded and durable joint according to the invention.

### Advantageous Effects of the Invention

The processes of the invention generate a seamless, materially bonded, homogeneous and at the same time mechanically stable, i.e. durable, connection between individual joining partners of tissue or one or more (free amino group-containing; collagen-containing) tissue component(s); for example, in the case of pericardium. The process enables a connection of two ends of one piece of tissue with each other or a connection of more two or more pieces of tissue with each other. A piece of tissue to be connected or joined preferably has an area of more than 0.5 mm²

The joining processes described and claimed above are all based on chemical crosslinking by a suitable crosslinking agent, such as glutaraldehyde. Since in the case of pericardial components, for example, this is in any case a mandatory process step for medical implants based thereon, the actual tissue connection/joining is thus realized without any additional material component in the end product, which is clearly a technical advantage of the disclosed processes. Beside the crosslinking agent (and the solvent of the crosslinking agent solution), e.g. glutaraldehyde or an aqueous solution of glutaraldehyde, no other chemicals may be required for the process using static and/or quasi-static and/or periodic pulsatile and vertical/orthogonal compressive loading/compression.

The use of these processes for medical applications significantly reduces the need for and number of surgical sutures/nodes in the case of implants based on, for example, collagen-containing tissue, as a result of which the sutures/nodes as the weak points described at the beginning are greatly reduced or at least eliminated at certain points, and at the same time the manufacturing costs of such implants are noticeably reduced.

The seamless, material bonded and mechanically resilient and durable connection/joint also makes it possible to achieve medical implants of smaller diameter, since, for example, the surgical sutures/nodes that would otherwise be necessary are no longer required.

Furthermore, it is possible to enclose alloplastic support structures/stents by the connections/joints of tissue/tissue components according to the invention in a seamless, material bonded and durable manner. In this regard, reference is made to the embodiments cited below; in particular, to an inflatable inner balloon or an inflatable cuff with an outer shape.

Advantageously, an average tensile shear strength (adhesive strength) of 14.82 cN (breaking load 7.4 N) could be achieved for stabilized tissue, for example (see embodiment examples below). Decisive influencing factors here, in addition to the initial condition of the tissue, are the compression type, cylinder force, overlap length and crosslinking time. In contrast, no significant influence can be determined for the choice of overlapping pericardial sides (see above).

In contrast to tensile stress, peel stress leads to failure of the material bond/joint already at a force of a few centi-newtons. In this case, the load-bearing crosslinks in the overlap area are unable to dissipate stress peaks, so that a brittle adhesive bond must be assumed. This lesson is given to the skilled person.

It should also be mentioned that the joining process according to the invention can possibly lead to a reduction in the water content in the tissue/component(s), which can possibly also affect the optical, structural and mechanical tissue properties. This may possibly lead to optically transparent overlap areas after the process.

### Examples of TAVI/TAVR Heart Valve Replacements and Covered Stents

For the following application examples, exemplary tissue components from porcine pericardium have been seamlessly, materially bonded, and durably bonded/joined in accordance with the processes of the invention. However, it is clearly evident to the skilled person that this process can be generally transferred to biological tissues and/or artificial tissues including free amino groups, which are correspondingly suitable for chemical crosslinking by, for example, glutaraldehyde.

### Examples A)—Tissue Processing

The starting material for the following experiments is porcine pericardium from approximately six-month-old pigs, which are obtained fresh from the slaughterhouse as required. During transport, the tissue is stored in isotonic saline (NaCl rinsing solution, sterile) at a mass concentration of 0.9% and initially cooled at 4 C for 1-2 hours prior to mechanical preparation.

Mechanical Preparation

In the first step of mechanical preparation, the pericardium is dissected along the pericardial cavity. Subsequently, the stable fibrous composite of pericardium fibrosum and lamina parietalis required for the heart valve replacement is freed from coarse fat and muscle remnants using surgical scissors or a scalpel. Adherent fatty tissue on the rough pericardial side can be wiped off with a compress soaked in saline solution. During the entire preparation process, the tissue must always be prevented from drying out in order to avoid irreversible damage to the tissue. After mechanical preparation, rinse the tissue three times in saline for 5 minutes to completely clean it.

Glutaraldehyde as an Exemplary Chemical Crosslinking Agent.

For crosslinking the tissue, a phosphate-buffered saline solution, DPBS for short (Dulbecco's Phosphate Buffered Saline w/o Ca and Mg), with a mass fraction of glutaraldehyde of 0.5% is always used in this work. For this purpose, 9 ml of a 50% glutaraldehyde solution is pipetted into pure DPBS per liter and dissolved in it.

Stabilization and Laser Cutting

A pulsed CO₂ laser (Epilog Zing 24; Epilog) with a maximum power of 30 W is used to shape the tissue. Cutting in the non-crosslinked tissue state requires, in addition to adjusting the laser power, additional stabilization and drying of the tissue, as the process can otherwise lead to significant internal stresses in the tissue and resulting high distortion of the samples. Without a preceding stabilization process, reproducible tissue cutting may not be possible.

A process for the preservation of biological tissues by controlled dehydration is used for stabilization. The three stabilization solutions used here are each composed of the stabilizer glycerol, PEG200 or PEG400 and ultrapure water with different mass fractions.

For the laser process in the non-crosslinked state, the tissue is rinsed for 15 minutes each in glycerol 30%, PEG200 40% and PEG400 40% after mechanical preparation and then dried in a suitable climatic chamber at 40° C. for a period of 12 hours, with the humidity being reduced linearly from 95% to 10%. The tissue is spread out on a ceramic plate, which can also serve as a base for the laser cut. Due to the low water content of the dried tissue, the laser power is reduced from 12% to 6% with otherwise unchanged conditions.

Uniaxial Tensile Tests and Thickness Measurement

In general, pericardial tissue is characterized by its viscoelastic material behavior. Despite the low thickness, this tissue shows a high mechanical load capacity and is elastic at the same time. Depending on the application of the tissue (e.g. as heart valve replacement material), it is sometimes exposed to high mechanical loads. Tensile tests to determine the mechanical properties are therefore a fundamental tool to assess the stability and stiffness of the tissue. The aim is always to process the tissue in such a way that mechanical integrity is maintained.

Uniaxial tensile tests to characterize the mechanical behavior of the biological tissue were performed on a test rig that allows both uniaxial and biaxial tensile tests. This measurement apparatus consists of four identical and independently controllable drive units. The clamping of the tissue to be tested (e.g. the seamlessly joined/connected tissue according to the invention) is realized by clamping jaws which are fixed to the carriage in a roller guide. Platform load cells with a measuring range of 0.01-85 N serve as force sensors.

Unless otherwise specified, a specimen geometry of 21 mm×3 mm is used for the tensile tests. The zero length of the tissue in the tensile test is determined automatically at a preload of 2 g. The travel speed of the jaws is, for example, 12 mm/min. In order to prevent the tissue from drying out, a Plexiglas tub is attached in the area of the clamping jaws, which is filled with isotonic saline solution during the measurement. In addition to the breaking force Fmax and the breaking strain εmax, the breaking stress σmax and the modulus of elasticity (Young's modulus) E can also be determined from the stress-strain diagram if the thickness of the specimen is known.

The thickness is measured tactilely with a circular measuring plunger (Ø 5 mm), which presses with a weight of 30 g for 2 s on the tissue to be tested. The arithmetic mean of three thickness measurements at different points on the specimen is always used.

Concept Description and Test Setup

The basic requirement for the formation of interfibrillar crosslinks is that the distance between the collagen fibers and microfibrils involved is smaller than the length of the molecules of the crosslinking agent, e.g. the glutaraldehyde molecules, which form the crosslink. The identification of suitable pressing parameters to reduce the fiber spacing is thus essential to enable a stable, seamless and material bonded bond by e.g. glutaraldehyde. At the same time, too high a pressing pressure (an excessive pressure load) during the quasi-static or periodic pulsatile pressure load/compression according to the invention potentially results in preventing accessibility of the crosslinking solution to the tissue/components to be bonded.

Therefore, not only static or quasi-static pressure on the tissue during crosslinking is possible, but also periodic pulsatile compression. In the pressureless phases during the crosslinking process, this ensures sufficient contact between the crosslinking agent (e.g. glutaraldehyde) and the tissue. For this purpose, a corresponding device has been developed according to the invention, with which both a quasi-static, relatively constant pressure and a more dynamic, periodic pulsatile pressure on the tissue/components can be generated.

The device (cf. FIG. 7) consists, for example, of two double-acting pneumatic cylinders (41), connected by solenoid valves, which are suspended vertically in a framework of aluminum profiles (39, 40).

The distance between the cylinders and the base plate (42) can be adjusted by telescopic locking sets (40). Each solenoid valve is connected to a double-acting cylinder via two connections.

The control thereby causes each valve to be either statically, quasi-statically or periodically-pulsatilically alternately open, resulting in a likewise static, quasi-static or periodically-pulsatilic retraction or extension of the cylinder piston rods in order to exert the essentially vertical pressure load/compression on the tissue(s) to be joined/connected. Both cylinders can be controlled independently, allowing two series of tests with different parameters to be performed simultaneously.

By controlling the solenoid valves and the applied air pressure, either a static, quasi-static or a periodic-pulsatile, defined force application essentially vertical/orthogonal to the base plate (42) of the assembly is thus generated for each cylinder. To generate a static or quasi-static, rather constant force input, the compressed air hose of the cylinder is directly connected to the compressed air source without an intermediate valve. The air pressure is continuously adjustable up to a maximum pressure. The force of an idealized piston is calculated according to:

F=p×A

Where p is the applied air pressure and A is the piston area of the cylinder. It should be noted that due to frictional effects, the effective piston force is about 10% less than the theoretical piston force. The support surface for the tissue samples is formed by exemplary laser-cut, cross shaped acrylic parts of 3 mm thickness. The two lateral holes are used to clamp the acrylic parts in an appropriately designed and 3D-printed holder made of polylactide. This allows the acrylic parts to be stacked exactly vertically, preventing horizontal movement. A stamp, also 3D-printed, is connected to the cylinder piston rod via a thread. This punch is used to transmit force between the cylinder and the specimen stack. At the same time, it prevents the acrylic parts from tilting sideways during the piston movement. By extending the cylinder piston rod, pressure/compression is exerted on the specimen stack (pressure phase/compression). When the cylinder is in the retracted state (pressure release/pressure pause), the chemical crosslinking solution used, e.g. 0.5% glutaraldehyde solution, easily reaches the tissue.

Seamless, Material Bonded and Durable Tissue Connection

According to the invention, a seamless, interlocking and durable connection/joining of tissue/tissue components is most preferable when a suitable crosslinking agent, such as glutaraldehyde, is involved in the process and forms crosslinks between the joining partners.

In the present embodiment example (see FIGS. 1 and 2), porcine pericardial tissues (1, 2) are first cut into rectangles (30 mm×10 mm) by laser and placed on the acrylic parts as a support surface (4) in such a way that an overlapping tissue area (3) of 10 mm×10 mm is formed between every two specimens (see FIG. 1). A rough pericardial side (pericardium fibrosum) is always brought to overlap with a smooth pericardial side (lamina parietalis). The overlapping tissue samples are additionally enclosed in laser-cut rectangular filter paper strips (50 mm×10 mm). The absorbency of the filter paper strips ensures accessibility of the crosslinking solution, in this case glutaraldehyde, to the tissue samples during the pressureless phases of periodic pulsatile pressure loading/compression.

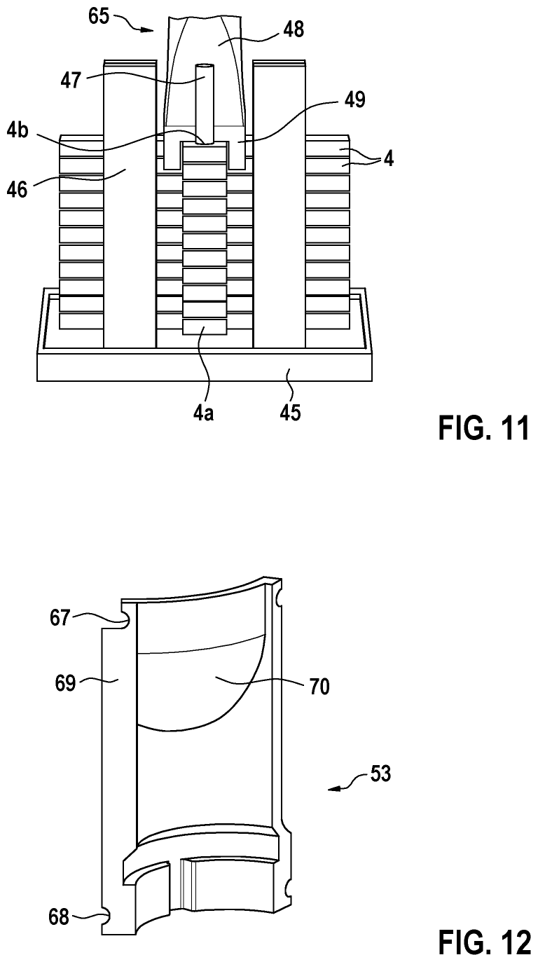

After the tissue is placed, ten acrylic parts covered with tissue and a final acrylic part are placed on top of each other on the 3D-printed holder (cf. FIG. 11). The holder with the tissue samples is then placed in a plastic vessel and aligned vertically under the punch (65) of the cylinder piston rod. The height of the pneumatic cylinder is adjusted via the telescopic locking sets so that the plunger rests on the uppermost acrylic part without pressure in the retracted cylinder state. The process according to the invention is started by connecting the system to the compressed air source and filling the plastic vessel with the crosslinking solution, in this case glutaraldehyde. For periodic pulsatile pressure loading/compression, the control system in this embodiment example is programmed such that the duration of pressure loading or pressure relief/pressure pause is 30 seconds each. The applied air pressure is controlled to 4.8 bar. This corresponds to a theoretical piston force of the cylinder of about 150 N. The curing time is set at 24 hours (1 day).

At the end of this total period of periodic pulsatile pressure/compression, the tissues are removed from the holder and transferred to saline as quickly as possible. To remove any residual unbound glutaraldehyde, the specimens are rinsed three times for five minutes in isotonic saline. Uniaxial tensile tests are used to check whether a seamless, materially bonded and durable connection/joint has been established between the tissue pieces. For this purpose, it is determined that tissue pieces are to be regarded as successfully joined if a short-term tensile shear load with a force of 1 N does not completely break the bond.

Results

The results of the tensile tests described above, among others, are plotted in Tab. 1 below. It can be seen that the joining/connecting of stabilized, non-crosslinked tissue by mere crosslinking with glutaraldehyde is possible in principle, but dynamic compressive loading/compression is required (quasi-static or periodic pulsatile) to generate a reproducible, stable joining/connecting. In contrast, both a mere static and dynamic pulsatile compression of the tissue samples with pure DPBS, i.e. without crosslinking agent, do not cause tissue bonding, as expected. Here, already the rinsing process of the tissues in saline leads to the failure of the bond in most cases.

In contrast, a significant influence of the compression type on the connection/joining of crosslinked tissue cannot be determined. Moreover, in this case, tissue bonding is in principle feasible both in a glutaraldehyde solution and in pure DPBS, but about half of the specimens fail at a force of 1 N already. This suggests that the glutaraldehyde molecules of the frame-crosslinked tissue, which are unilaterally bound in the tissue, generally have the ability to form interfibrillar crosslinks. It can be assumed that this is not only explained by direct binding of the unilaterally bonded glutaraldehyde molecules to free amino groups of the joining partner, but is largely due to the polymerization of the glutaraldehyde between the joining partners.

Overall, these results confirm the theory that the formation of interfibrillar crosslinks by glutaraldehyde between two pieces of tissue is possible in principle and that only in this way can a sutureless, material bonded and durable joining of pericardial tissue be achieved.

TABLE 1
Results of sutureless, material bonded and durable tissue bonding/joining
according to the embodiment example explained above.

| | | Number of successful | |
|---|---|---|---|
| Tissue- | Compression | Tissue connections | |
| condition | type | DPBS | Glutaraldehyde solution |
| stabilized | statisch | 0/10 | 4/10 |
| | pulsatil | 0/10 | 10/10 |
| crosslinked | statisch | 6/10 | 5/10 |
| | pulsatil | 5/10 | 6/10 |

Influence of the Crosslinking Time

The measurement results with variation of the curing time are listed in Table 2 below. Tripling the curing time from one day to three days increases the breaking load and shear strength by about 65%.

TABLE 2
Influence of the crosslinking time on the adhesive strength, thickness and
crosslinking quality in the overlap and edge areas (indicated in brackets)
in the joining of pericardial tissues according to the invention (n = 10).

| Crosslinking time | $\begin{matrix}Adhesive ‐ \\ strength \\ in \frac{cN}{{mm}^{2}}\end{matrix}$ | Denaturation- temperature in ° C. | $\begin{matrix}Amino ‐ \\ groups \\ in \frac{µg}{mg}\end{matrix}$ | Thickness- coefficient |
|---|---|---|---|---|
| 1 day | 9.0 ± 1.7 | 96.0 ± 0.7 | 5.7 ± 1.4 | 1.66 ± 0.23 |
| | | (93.4 ± 0.3) | (5.9 ± 1.2) | |
| 3 days | 14.8 ± 3.8 | 96.5 ± 0.7 | 4.7 ± 0.5 | 1.73 ± 0.25 |
| | | (93.2 ± 0.9) | (5.1 ± 0.8) | |
| 8 days | 14.7 ± 2.1 | 96.9 ± 0.7 | 4.6 ± 0.7 | 1.72 ± 0.25 |
| | | (94.3 ± 1.0) | (5.1 ± 1.7) | |

Together with the results of the amino group detection, the following theory can be put forward: In the first 24 hours, the majority of free amino groups are occupied by glutaraldehyde oligomers. The probability for the formation of interfibrillar crosslinks is maximal during this period. Subsequently, the number of free amino groups decreases only insignificantly, and the polymerization of glutaraldehyde dominates during this period. Unilaterally bonded as well as free glutaraldehyde oligomers combine and form additional load-bearing interfibrillar bonds between the tissue joining partners, which are reflected in a significant increase in the breaking strength. A further increase in the crosslinking time is ineffective in that no more load-bearing bonds are generated by polymerization either.

Within the scope of the invention, parameters could be determined as optimal with which overlapping tissues/tissue components can be joined in a reproducible, seamless, material bonded and stable manner. With the process parameters listed in Tab. 3, an average shear strength of 14.8 cN/mm² can be achieved. This corresponds to a breaking load of the overlap of 7.4 N. Frame-crosslinked reference tissue (stabilized and dried before the start of crosslinking) has an average breaking load of 19.7 N (n=30) for the same crosslinking time and a tissue width of also 10 mm. This means that chemical crosslinking of overlapping tissue samples can generate a joint/joint whose breaking force (for an overlap area of 50 mm²) under tensile shear stress corresponds to about 38% of the breaking force of conventional tissue.

TABLE 3
Process parameters for maximizing the shear strength
in the seamless, materially bonded and durable joining
of overlapping tissue areas/tissue components

| Process parameters | Determined optimum | |
|---|---|---|
| Pressure change times | 30 s/2 s | |
| Cylinder force | 225N | |
| Tissue condition | stabilized | |
| Overlapping pericardial sides | smooth-smooth | |
| Overlapping length | 5 | mm |
| Crosslinking time | 3 | days |

Optical Properties and Surface Topography

The effects of the bonding process of overlapping tissue samples on the collagen fiber structure were analyzed, for example, in this embodiment. First, a visual inspection was performed, focusing on the influence of pressure on the optical properties of the tissue. This is followed by a detailed examination of the surface topography under a scanning electron microscope. Of particular interest here is the extent to which the bonding process of the tissue influences the arrangement of the collagen fibers in the overlap and edge regions. For the preparation of the tissue samples, the optimal process parameters for tissue bonding are used according to Table 3 (see above).

Even without optical aids, the influence of the joining process on the optical properties of the tissue can be clearly seen. Compared to frame-crosslinked tissue, the tissue samples bonded under pressure (quasi-static or periodic pulsatile) are almost completely transparent in the overlap area as well as in the single-layer tissue area. Individual fibers are not discernible. The rough tissue side (pericardium fibrosum) is visually indistinguishable from the smooth tissue side (lamina parietalis), and the overlap region is also barely visually distinguishable from the single-layer tissue region. The increased transparency of the tissue can basically be explained by the removal of water. In frame-crosslinked tissue, there is free or bound water between the individual collagen fibers. At each interface between collagen (refractive index 1.4-1.55) and water (refractive index 1.3), light is refracted as it passes through the tissue. Due to the inhomogeneous distribution of collagen in pericardium, a chaotic refraction pattern results, and the tissue appears opaque. Pressure loading/compression according to the invention forces the interfibrillar water out of the tissue, so that the number of interfacial junctions decreases and the transparency of the tissue increases.

Mechanical Properties and Water Content

The transparency of the tissue already indicates that the quasi-static or periodic pulsatile compression significantly decreases the amount of interfibrillar water. In the following embodiment example, it will be shown to what extent this affects the mechanical properties of the tissue. In addition to the breaking stress, the elongation at break and the modulus of elasticity, the water content of the tissue is determined. To produce the tissue samples bonded according to the invention, the optimum process parameters from Table 3 are used and applied as described above. Sampling for uniaxial tensile tests is performed in the single-layer tissue section. The water content is measured differentially for the overlap area as well as the single-layer tissue area. Frame-crosslinked tissue, which was also subjected to a stabilization and drying process before the start of the three-day crosslinking process, serves as a reference.

Uniaxial Tensile Tests

The results of the uniaxial tensile test are shown in Table 4 below. Obviously, the periodic pulsatile compressive loading/compression in particular causes a significant thickness reduction of the tissue. The nearly identical breaking forces of the compressed tissue and the reference tissue suggest that the collagen fibers are not damaged by the compaction process. Due to the increased density of the collagen fibers, the breaking stress and Young's modulus increase considerably. At the same time, a much lower elongation at break is observed compared to the frame crosslinked reference tissue. It can be assumed that the individual fibers do not lose their load-bearing capacity as a result of the pressing process, but that the sliding of the collagen fibers against each other is impeded by dehydration.

TABLE 4
Mechanical properties and thickness of the tissue crosslinked
under periodic pulsatile compression in the single-layer
tissue section (composite single-layer): frame-crosslinked
tissue serves as reference. (n = 30)

| | | Breaking | Breaking | Elongation | Young's |
|---|---|---|---|---|---|
| Sample | | force | stress | at break | modulus |
| type | Thickness | in N | in MPa | in % | in MPa |
| Reference | 0.07 ± | 4.4 ± | 22.1 ± | 22.7 ± | 150.3 ± |
| | 0.01 | 1.7 | 8.4 | 3.2 | 62.4 |
| Composite | 0.05 ± | 4.5 ± | 30.5 ± | 16.3 ± | 255.3 ± |
| single layer | 0.01 | 1.5 | 8.5 | 3.6 | 82.3 |

Water Content

The results of water content determination in Table 5 confirm the structural changes of the tissue due to crosslinking under periodic pulsatile compression. Compared to frame-crosslinked tissue, the water content is much lower in both the single-layer tissue and the overlap region.

In principle, there are two explanations for this: First, it can be assumed that the water removal is not limited to unbound water molecules. If bound water molecules are also removed from the tissue, it can be assumed that hydrogen bonds cause embrittlement of the tissue and preclude complete rehydration. Second, it is conceivable that the higher collagen fiber density increases the likelihood of interfibrillar crosslinks due to reaction with glutaraldehyde. That is, the mechanism used to join two tissue samples also leads to adhesion of the collagen fiber layers within the individual tissue samples and in this way impedes the reincorporation of water molecules.

TABLE 5
Water content of tissue crosslinked under periodic pulsatile compressive
loading/compression in the single-layer tissue region (composite
single-layer) and overlap region (composite overlap): frame crosslinked
tissue serves as reference. (n = 10)

| Sample type | Water content in % |
|---|---|
| Reference | 74.3 ± 0.9 |
| Composite single layer | 55.2 ± 2.6 |
| Composite overlap | 53.8 ± 2.8 |

### Summary of Embodiments A)

In the above-mentioned embodiments, a process (essentially the periodic-pulsatile variant) for seamless, material bonded and durable joining was illustrated using pericardial tissue. According to the invention, a pneumatic assembly was used as an exemplary device to achieve both static, quasi-static and periodic-pulsatile pressure loading/compression of overlapping tissues/tissue regions/tissue components can be achieved. In the above-mentioned embodiments relating to the tissue per se, it was shown that the joining process according to the invention is fundamentally based on the formation of interfibrillar crosslinks between the joining partners. Accordingly, a suitable chemical crosslinking solution, preferably glutaraldehyde solution, is always required far preferentially for joining non-crosslinked, stabilized tissues. In contrast, pre-crosslinked tissues can in principle be joined even in pure DPBS. It can therefore be assumed that the bonding mechanism is not exclusively due to the direct, bilateral bonding of free glutaraldehyde oligomers between the joining partners, but also to the polymerization of unilaterally bonded glutaraldehyde molecules in the overlap region.

With optimum parameters, an average tensile shear strength (adhesive strength) of 14.82 cN/mm² (breaking load: 7.4 N) could be achieved for stabilized tissue according to the invention. In addition to the initial condition of the tissue itself, the compression type (quasi-static or periodic-pulsatile), cylinder force, overlap length and crosslinking time should be mentioned as decisive influencing factors.

Furthermore, it was found that the processes according to the invention lead to a noticeable reduction of the water content in the tissue, which has a massive effect on the optical, structural and mechanical tissue properties. For example, the joints produced in the above-mentioned embodiment examples are almost completely transparent in the overlap area as well as in the single-layer edge area. Although the collagen fibers are not destroyed by the pressure load/compression of the tissue and the associated reduction in thickness, their freedom of movement is considerably restricted. As a result, the breaking stress and modulus of elasticity increase, and the elongation at break is reduced.

Overall, the processes according to the invention offer an applicable technical solution for seamless, material bonded and durable bonding of tissues including free amino groups, in particular tissues containing collagen. However, the application of these processes must always take into account the load limits of the joint in different load cases, as well as the altered mechanical and structural tissue properties due to the compression/compression process.

### Examples of Embodiments B)—Suture Reduction in a TAVI/TAVR Valve

Using an exemplary self-expanding TAVI/TAVR valve, the following demonstrates how the processes according to the invention can be integrated into the manufacturing process of a cardiovascular implant. The aim is to achieve a reduction in the number of surgical knots/sutures by bringing about one or more sutureless connections/joints of one or more tissue component(s) of a TAVI-TAVR valve—without compromising the functionality of the valve prosthesis.

After mechanical preparation (see above), the pericardial tissue is first stabilized and dried in a climate chamber. The tissue is then cut with a suitable laser in such a way that a defined overlap of the tissue ends of what is in this case a one-piece tissue component is created by placing it on a suitable mold (FIG. 3). This is located exclusively in the skirt area, so that the function of the leaflets is unaffected.

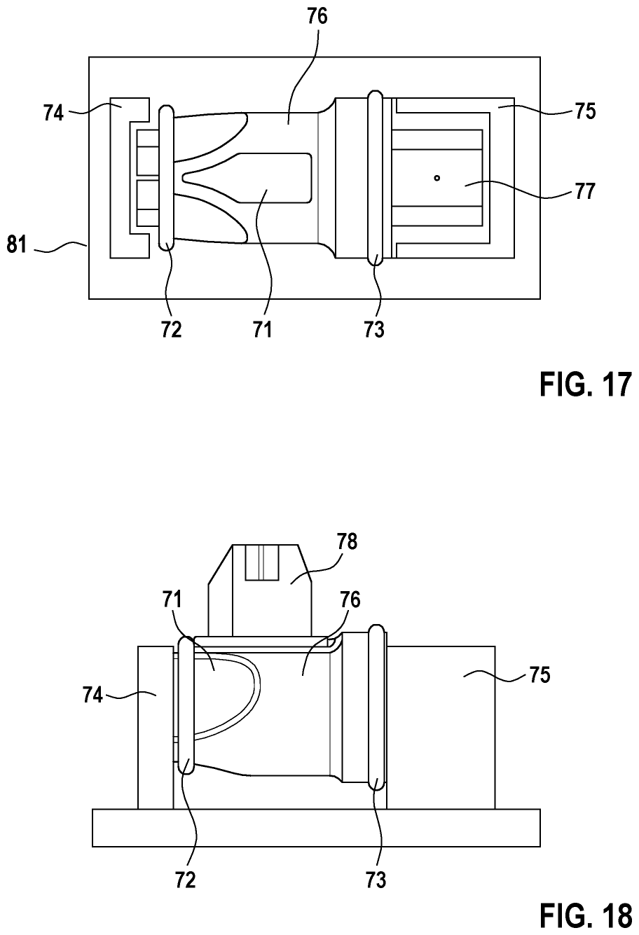

After the tissue has been clamped in a forming structure (FIGS. 17, 18), the chemical crosslinking process begins; in this case with glutaraldehyde solution. During the crosslinking with glutaraldehyde, the overlap area is periodically pulsatilized with pressure load by a punch (78) adapted to the recess in combination with the device according to the invention in order to realize the seamless connection of the tissue ends.

After the crosslinking process, the side parts are removed and the excess tissue is removed by the second laser process on the molded body. In the last step, the valve is connected to the stent, equivalent to the conventional manufacturing process. That is, the goal of this embodiment is to avoid any suturing of the tissue component per se; however, the sutures for placement/fixation to the stent remain.

To realize the integral connection of the tissue ends during the three-dimensional crosslinking process, a molding construction is provided as described below. The aim of the design is to enable vertical force/pressure to be applied to the overlap area(s), while at the same time fixing the tissue to the molded body during the crosslinking process. For this purpose, both the side parts and the molded body are modified, and a holder and a punch are also provided, which is used to transfer force from the pneumatic cylinder to the overlap area of the tissue.

In the case of two of the three side parts, a recess is created in the web area instead of the extension; this recess is precisely matched to the dimensions of the punch and serves as a guide for it. The punch is connected via a thread to the pneumatic cylinder of the device for periodic pulsatile pressure loading/compression and is adapted to the curvature of the molded part. A corresponding support prevents tilting of the molded body during periodic pulsatile force application.

Tissue Processing

For the fabrication of the exemplary TAVI/TAVR valve, the pericardial tissue is first stabilized after mechanical preparation and dried in a climate chamber. A suitable cutting pattern is used for the first laser cutting process.

A suitable tissue geometry makes it possible to place the tissue on the molded body without wrinkles and at the same time generate a defined, overlapping tissue area. This has a width of 10 mm in the skirt area and tapers to a minimum width of 1.2 mm between the leaflets. A reproducible lay-up of the tissue in its native state is not recommended, as it tends to wrinkle in the edge area. In this case, too, the molded part is wrapped with self-adhesive aluminum foil to prevent it from being damaged by the subsequent laser process.

After tissue placement, the side parts are attached so that the recesses created enclose the overlap area. To fix the side parts, rubber rings are attached to the grooves provided. The assembled structure is then inserted into the holder with the overlapping tissue area facing upwards. An appropriately cut filter paper strip is placed on the overlap area to promote accessibility of the glutaraldehyde solution to the tissue. An additional thin silicone pad has proven effective to ensure homogeneous pressure distribution over the entire overlap area. The edges of the recess serve as a guide for the plunger, which is positioned via the telescopic locking sets so that it rests on the silicone layer without pressure when the cylinder plunger is retracted. Three-day crosslinking using glutaraldehyde is performed with a periodic pulsatile pressure load/pressure pause of the cylinder at a ratio of 30:2 seconds; i.e., 30 seconds of pressure load/compression per cycle and 2 seconds of pressure pause per cycle. The theoretical cylinder force is 150 N, which corresponds to a pressure of 0.71 N/mm² in the overlap area. The subsequent laser process in combination with the turning device gives the tissue its final shape.

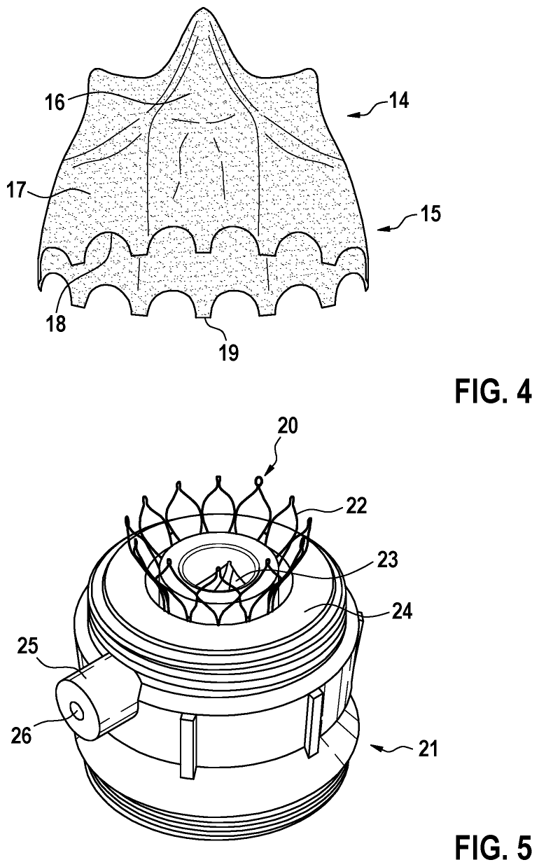

Due to the seamless, material bonded and durable connection of the tissue ends of the TAVI-TAVR valve tissue component obtained, only the cutting of the e.g. three leaflet and the e.g. twelve skirt sheets is required according to the invention. The tissue can then be carefully pulled off over the three-sided prism of the molded body. The result of the tissue processing is shown in FIG. 4. Thus, only a simplified suturing process is used for suturing the TAVI/TAVR tissue component(s) into the stent, since the suturing of individual tissue components to each other, such as individual leaflet and skirt components, is omitted.

In another embodiment, an approach is disclosed for achieving a sutureless connection of the tissue component(s) of a TAVI/TAVR valve to the stent.

In addition to the classic internal skirt component, aortic valve implants optionally contain another pericardial strip attached to the outside of the stent (external skirt). This additional border serves to reduce paravalvular leakage (PVL) and is crucial for the approach described below.

The basic idea is to generate a sutureless connection between inner and outer skirt part with stent in between.

Mechanical preparation and stabilization and drying of the pericardial tissue is followed by a two-part laser process. First, the inner tissue component is cut. In addition, a second tissue component is cut out that corresponds to the skirt area of the first tissue component. The tissue components are then placed in an inflation sleeve device so that the stent is enclosed from both sides in the skirt area and an overlap area is formed between the stent struts.

Radial compression is thus required to generate a circumferential connection between the inner and outer skirt components. According to the invention, an annular, double-walled silicone sleeve (inflation sleeve device) is provided specifically for this purpose, which inflates radially inward in a time-dependent manner Thus, a homogeneous, quasi-static or periodic pulsatile pressure load/compression of the tissue in the overlap area is achieved and in this way a seamless connection of the tissue component to both sides of the stent (inner and outer side) is realized.

For reproducible production of an inflatable cuff in the sense of the invention with a defined wall thickness, the two-part mold shown in FIG. 5 was designed.

Four symmetrically arranged holes are provided in the otherwise closed bottom of the lower casting for subsequent demolding. These are closed with screws before the casting process begins in order to produce a flush bottom surface. For the casting process, a 3D-printed hollow cylinder made of water-soluble polyvinyl alcohol (PVA) is first suspended symmetrically in the mold via additionally designed and 3D-printed PLA rods so that a defined gap dimension is created between the hollow cylinder and the mold on each side. Corresponding holes in the mold as well as recesses in the PVA hollow cylinder are provided for correct positioning of the rods. At the beginning of the two-stage casting process, the mold is half filled with silicone. After the silicone has cured, the PLA rods are moved outward until they also flush the wall of the mold. At this point, the PVA hollow cylinder is self-supportingly embedded in the silicone compound. The entire mold is then filled with silicone up to a designated edge on the lid so that the PVA core is completely enclosed. During the entire casting and curing process, an outwardly directed compressed air hose is attached to the side of the PVA hollow cylinder in a further recess. This is also embedded in the silicone compound through a bulge in the two-part mold.

After curing of the silicone compound and demolding of the sleeve, the water-soluble PVA core is finally washed out. This is designed to give a wall thickness of 3 mm for the outer wall of the inflatable sleeve and for the base and lid. The thickness of the inner wall is set at 4 mm, since the sleeve is exposed to the highest stresses in this area during the crosslinking process. The finished, double-walled inflation sleeve is shown in FIG. 5.

A suitable control system in combination with a solenoid valve is used to control the inflation process.

The starting material for this embodiment is two mechanically prepared, stabilized and dried tissue patches, which are first processed with the laser to provide a suitable cutting geometry.

The sequence of subsequent tissue placement includes the steps of rolling the first tissue component onto the lower, thin-walled support structure so that the leaflets are freely supported for movement.

Subsequently, the shape memory effect of the nitinol is exploited to achieve the correct placement of the stent. For this purpose, the support structure including the tissue is centered on the guide plate. Meanwhile, the stent is radially expanded in ice water with an auxiliary body and then swiftly slipped over the tissue-covered component.

While the stent regains its original geometry upon heating to room temperature, the gaps on the guide plate ensure proper alignment of the stent relative to the support structure. The outer skirt is then rolled flush on the outside of the stent. The tissue components are then sutured to the eyelets of the stent. The individual surgical knots do not serve to connect to the stent, but are essential for proper alignment of the tissue components to each other. Subsequently, the remaining support components are assembled into a hollow cylinder with the leaflets facing inward through appropriately provided gaps.

Essentially, tissue placement to join the inner and outer skirts includes the following steps:
Placement of the inner tissue component onto the lower support structure.
Center placement of the lower support structure on the guide plate
Placement of the nitinol stent
Placement of the outer tissue component
Removal of the prefabricated, sutured valve prosthesis
Assembly of the final construction.

After the tissue placement described above, the experimental construct is assembled as follows: First, the inflatable sleeve is inserted into the lower specimen chamber so that the compressed air port occupies the designated lateral hole. The prefabricated valve together with the support cylinder is enclosed with a suitably cut rectangular filter paper strip and then sunk centrally into the sample chamber. In order to prevent vertical displacement of the valve during the compression process, the thin-walled lower support structure is firmly screwed to the sample chamber via holes provided.

Now the inflation collar is connected to the compressed air hose of the solenoid valve and the control system is connected. The periodic-pulsatile pressure-change time for this embodiment is set at 30:2 seconds; i.e., 30 seconds of pressure load/compression per cycle and 2 seconds of pressure pause per cycle. To avoid failure of the inflation cuff, the pressure is controlled at 2 bar. Due to the liquid-like behavior of the silicone, the inflation of the sleeve generates a pressure greater than 0.1 N/mm² inside the specimen chamber.

After three days of crosslinking in this case (total duration of crosslinking) under periodic pulsatile radial compression, the valve prosthesis is demolded. Radial expansion of the stent in the skirt region to remove the support structures is not appropriate here, as this potentially damages the adhesive bond. While the bottom plate as well as the upper support structure and the thick-walled inner core can be easily removed, an additional process step is therefore necessary to remove the thin-walled inner core. For this purpose, the structure is heated to 70° C. in a water bath. This temperature is above the softening temperature of the thin-walled PLA component, so that it can be plastically deformed without high force. At the same time, the denaturation temperature of the crosslinked tissue is not exceeded. This makes it possible to detach the support structure from the construction without changing the conformation of the collagen fibers and damaging the adhesive bond.

Thus, in the latter embodiment, a concept according to the invention was illustrated, which for the first time enables a seamless connection of the tissue component to the stent by generating a tissue connection between the inner and outer skirt with the stent in between.

Overall, it can be seen that the seamless, interfacing and durable tissue connection of the invention can be profitably integrated into the manufacturing process of e.g. TAVI/TAVR valves without compromising the functionality of the implant.

### Example C)—Biological Stent Graft

In the context of the invention, a biological stent graft may be prepared as follows:
(1) A layer of tissue is wrapped around an outer surface of a stent graft and folded over inwardly;
(2) A balloon that can be dilated inside the stent is inserted;
(3) The stent is wrapped on its outer side with a technical fabric as an interlayer;
(4) A cylindrical perforated outer shape, i.e., with holes, is mechanically fixed on the outside;
(5) Dilatation of the inner balloon with a static pressure;
(6) Fixation of the tissue of the stent framework in 0.5% glutaraldehyde for three days;
(7) Demolding/removal of a stent seamlessly encased with tissue.

The embodiments described herein are primarily for exemplary illustration of the invention. The number and/or design of compressive loads/compressions of the tissues to be joined/connected in the presence of a suitable crosslinking agent (particularly with respect to the concentrations and compositions thereof of the crosslinking agent solution) may be identified as suitable and varied by the skilled person within the scope of his knowledge.

With reference to the above disclosure, the present invention further includes the embodiments numbered in ascending order below:

A. Process for seamless (material bonded and durable) joining or connecting of tissue or one or more tissue component(s) (1, 2, 7), preferably (substantially non-crosslinked) tissue or tissue component(s), for medical applications, in particular for use as a component of a medical implant, preferably a vascular implant, more preferably an artificial heart valve (27, 28) or a covered stent, the process including at least the following steps:
a) providing one or more tissue(s) to be connected or joined (1, 2, 7), which may have or form one or more overlap region(s) (3);
b) providing a suitable container, mold and/or support surface for the tissue component(s) (4, 11, 12, 13, 53, 54);
c) providing a device (37, 38) capable of receiving the container, mold and/or support surface (4, 11, 12, 13, 53, 54) in a form-fit manner, and further capable of providing controllable static and/or quasi-static and/or periodic-pulsatile and (substantially) vertical or orthogonal compressive loading or compression of said overlap area(s) (41, 42);
d) optional cutting of the tissue or tissue component(s) (1, 2, 7) to be joined or connected by a suitable cutting instrument and/or a suitable cutting device;
e) placing or arranging the tissue or tissue component(s) according to step a) or d) in the container, in the mold and/or on the support surface according to step b) (4, 11, 12, 13, 53, 54) in such a way that the overlapping areas (3) intended for joining or connecting come to lie on top of one another in the device according to step c);
f) chemical crosslinking of the tissue(s) or tissue component(s) according to step e), in particular of said overlap areas, in the device according to step c) with addition of a suitable crosslinking agent into the container, into the mold and/or onto the support surface and under the action of said static, quasi-static or periodic pulsatile pressure load or compression on the overlap area(s) (41, 42);
g) demolding or removal of the tissue or tissue component(s) bonded or joined after step f) (6);
h) optional (purely) chemical post-crosslinking using a suitable crosslinking agent.

B. The process according to embodiment A, wherein the process includes at least the following steps:
a) providing one or more tissue(s) or tissue component(s) (1, 2, 7) to be joined or connected, preferably (substantially non-crosslinked) tissue(s) or tissue component(s), which may have or form one or more overlap area(s) (3);
b) providing a suitable container, mold and/or support surface for the tissue/tissue component(s) (4, 11, 12, 13, 53, 54);
c) providing a device (37, 38) capable of receiving said container, mold and/or support surface (4, 11, 12, 13, 53, 54) in a form-fit manner and further capable of providing controllable static and/or quasi-static and/or periodic-pulsatile and (substantially)vertical/orthogonal compressive loading or compression of said overlap area(s), wherein the force input of said compressive loading or compression is exerted in a range of 0.01 N/mm² to 10 N/mm² (41, 42);
d) optional cutting of the tissue or tissue component(s) (1, 2, 7) to be joined/connected by a suitable cutting instrument and/or a suitable cutting device;
e) placing or arranging the tissue or tissue component(s) according to step a) or d) in the container, in the mold and/or on the support surface according to step b) (4, 11, 12, 13, 53, 54) in such a way that the overlapping areas (3) intended for joining/connecting come to lie on top of one another in the device according to step c);
f) chemical crosslinking of the tissue(s) or tissue component(s) according to step e), in particular of said overlap areas, in the device according to step c) with addition of a suitable crosslinking agent into the container, into the mold and/or onto the support surface according to step b) and under the action of said static or quasi-static or periodic pulsatile pressure load or compression on the overlap area(s) (41, 42);
g) demolding or removal of the tissue or tissue component(s) bonded/joined after step f) (6);
h) optional (purely) chemical post-crosslinking using a suitable crosslinking agent.

C. The process according to embodiment A or B, wherein the process includes at least the following steps:
a) providing one or more tissue(s) or tissue component(s) (1, 2, 7) to be joined or connected, preferably (substantially non-crosslinked) tissue(s) or tissue component(s) including crosslinkable groups, and which is may have or form one or more overlap areas (3);
b) providing a suitable container, mold and/or support surface for the tissue/tissue component(s) (4, 11, 12, 13, 53, 54);
c) providing a device (37, 38) capable of receiving said container, mold and/or support surface (4, 11, 12, 13, 53, 54) in a form-fit manner and further capable of providing controllable static and/or quasi-static and/or periodic-pulsatile and (substantially) vertical or orthogonal compressive loading or compression of said overlap area(s), wherein the force application of the pressure load or compression is applied in a range of 0.01 N/mm² to 10 N/mm², preferably 0.1 N/mm² to 1 N/mm², over a time in the range of 1 second to 15 minutes in combination with a corresponding pressure release or pressure pause of 1 to 60 seconds, and this over a total period of at least 4 hours up to a maximum of 12 days (41, 42);
d) optional cutting of the tissue or tissue component(s) (1, 2, 7) to be joined or connected by a suitable cutting instrument and/or a suitable cutting device;
e) placing or arranging the tissue or tissue component(s) according to step a) or d) in the container, in the mold and/or on the support surface (4, 11, 12, 13, 53, 54) in such a way that the overlapping areas intended for joining or connecting come to lie on top of one another in the device according to step c);
f) chemical crosslinking of the tissue(s) or tissue component(s) according to step e), in particular of said overlap areas, in the device according to step c) with addition of a suitable crosslinking agent into the container, into the mold and/or onto the support surface and under the action of said static or quasi-static or periodic pulsatile pressure load/compression on the overlap area(s) (41, 42);
g) demolding or removal of the tissue or tissue component(s) bonded or joined after step f) (6);
h) optional (purely) chemical post-crosslinking using a suitable crosslinking agent.

D. The process according to any of embodiments above, wherein the static compressive load/compression is applied via a force application in the range of 0.01 N/mm² to 10 N/mm², preferably 0.1 N/mm² to 1 N/mm², over a total period of at least 4 hours to 5 days, preferably 2 days.

E. The process according to any one of embodiments above, wherein the quasi-static pressure load/compression is applied via a force application in the range of 0.01 N/mm² to 10 N/mm², preferably 0.1 N/mm² to 1 N/mm², over a time in the range of 200 seconds to 400 seconds, preferably 300 seconds, in combination with a corresponding pressure release/pressure pause of 1 to 10 seconds, preferably 1 or 2 seconds, and this over a total period of at least 4 hours to 5 days, preferably 2 days.

F. The process according to any of embodiments above, wherein the periodic pulsatile pressure load/compression is applied over a force input in the range of 0.01 N/mm² to 10 N/mm², preferably 0.1 N/mm² to 1 N/mm² over a time in the range of 10 seconds to 60 seconds, preferably 30 seconds, in combination with a corresponding pressure release/pressure pause of 1 to 10 seconds, preferably 1 or 2 seconds, and this over a total period of at least 4 hours to 5 days, preferably 2 days. 7. process according to any of embodiments 1 to 6, wherein the device according to step c) includes an electronically controllable pneumatic cylinder (41), hydraulic cylinder or inflation sleeve (21), which can be controlled via a suitable control element including suitable electronics in such a way that said quasi-static or periodic pulsatile pressure/compression movement can act on the overlap area(s) of the tissue/component(s) (3) (substantially) vertically/orthogonally.

G. The process according to any of embodiments above, wherein the crosslinking agent is an aldehyde-containing solution or is selected from the group consisting of glutaraldehyde, carbodiimide, formaldehyde, glutaraldehyde acetals, acyl azides, cyanimide, genipin, tannin, pentagalloyl glucose, phytate, proanthocyanidin, reuterin, and/or contains epoxy compounds.

H. The process according to any of embodiments above, wherein the crosslinking agent is glutaraldehyde, preferably a 0.5% to 0.65% glutaraldehyde solution.

I. The process according to any of embodiments above, wherein the tissue/component(s) has been subjected to a pretreatment including optional decellularization with a suitable detergent, preferably with a solution containing surfactin and deoxycholic acid, and optionally pre-crosslinking, preferably with a solution containing glutaraldehyde.

J. The process according to any one of embodiments above, wherein the tissue/tissue component(s) is rinsed at least once with a suitable solution, in particular a salt solution and/or an alcohol solution, before and/or after the crosslinking, the optional pre-crosslinking and/or the optional post-crosslinking.

K. The process according to any of embodiments above, wherein the process further includes performing a structural stabilization step on the, optionally decellularized, tissue/component(s) before or after the crosslinking, the optional pre-crosslinking and/or the optional post-crosslinking.

L. The process according to embodiment K, wherein the structural stabilization step is performed on the, optionally decellularized, tissue/tissue component(s) after crosslinking, after optional pre-crosslinking, or after optional post-crosslinking.

M. The process according to any of embodiments K or L, wherein the structure stabilization step includes exposing the, optionally decellularized, tissue/component(s) to at least one solution, but preferably at least two different solutions, wherein one solution includes glycerol and another solution includes polyethylene glycol.

N. The process according to embodiment M, wherein exposure to one or more of the solutions lasts from 5 minutes to 2 hours.

O. The process according to any of embodiments above, further including drying the tissue/tissue component(s) in a suitable controllable environment, such as a climatic chamber or desiccator, for example at constant low relative humidity or by reducing the relative humidity, optionally from 95% to 10% over 12 hours at 37° C.

P. The process according to any one of embodiments K to O, wherein of the at least two different solutions, a first aqueous solution includes polyethylene glycol having an average molecular weight between 150 g/mol and 300 g/mol; and a second solution is an aqueous solution of polyethylene glycol having an average molecular weight between 200 g/mol and 6000 g/mol.

Q. The process according to any one of embodiments K to P, wherein of the at least two different solutions, a first solution includes aqueous polyethylene glycol having an average molecular weight between 200 g/mol and 600 g/mol; and a second solution is an aqueous solution of polyethylene glycol having an average molecular weight between 200 g/mol and 6000 g/mol.

R. The process according to any one of embodiments above, wherein the process additionally includes removing alpha-gal epitopes by using a suitable alpha-galactosidase.

S. The process according to embodiment R, wherein the alpha-galactosidase is obtained from green coffee bean (GCB).

T. The process according to embodiment R, wherein the alpha-galactosidase is obtained from Cucumis melo.

U. Use of an aldehyde-containing crosslinking agent or a crosslinking agent selected from the group consisting of glutaraldehyde, carbodiimide, formaldehyde, glutaraldehyde acetals, acyl azides, cyanimide, genipin, tannin, pentagalloyl glucose, phytate, proanthocyanidin, reuterin, and/or epoxy compound-containing crosslinking agents in combination with a static or quasi-static or periodic pulsatile compressive load/compression to form interfibrillar crosslinks for seamless, material bonded and durable joining/connecting of tissues and/or one or more tissue component(s) for medical applications.

V. The use according to embodiment U, wherein the crosslinking agent is glutaraldehyde.

W. Seamlessly joined or connected tissue or tissue component(s) obtained according to any of the processes according to embodiments A to T for medical application, in particular for application in vascular implants, preferably in an artificial heart valve or in a covered stent.

X. Medical implant, preferably with a hollow cylindrical base structure, wherein in and/or on a surface of the base structure the seamlessly, materially and permanently connected/joined tissue or tissue component according to embodiment W is arranged/fixed, and which in the implanted state of the medical implant is intended and arranged to contact an anatomical structure of a patient, in particular a vessel wall, in particular a vessel, to which the medical implant has been implanted.

Y. The medical implant according to embodiment X, wherein the implant is a prosthetic heart valve including an artificial heart valve made of said tissue/component and/or a seal made of said tissue/component, which is attached, preferably sutured, to an expandable or self-expanding and catheter implantable base body.

Z. The medical implant according to any of embodiments X or Y, wherein the medical implant is selected from the group consisting of an artificial heart valve, in particular an artificial aortic valve, a coronary or peripheral vascular stent, in particular a covered stent and/or a stent graft.

AA. The medical implant according to embodiment X, wherein the tissue/tissue component is selected from the group consisting of pericardium, ligaments, tendon, cartilage, bone, skin, native biological tissue, autologous tissue, xenogeneic tissue, allogeneic tissue or collagen containing tissue.

BB. Medical implant including at least one seamlessly joined or connected tissue or tissue components according to embodiment W or obtained by a method according to any one of the embodiments A to R.

CC. The medical implant according to embodiment BB, wherein the medical implant is a cardiovascular implant, a endovascular prostheses, an endoprostheses, an esophageal implants, a bile duct implant, a dental implant, an orthopedic implant, a sensory implant, a neurological implant a microchip containing implant.

DD. The medical implant according to embodiment BB, wherein the medical implant is a stent, a vascular stent, a drug eluting stent, a pulmonary valve stent, a bile duct stent, a peripheral stent, a mitral stent, a stent graft, a venous valve, a tooth implant, a bone implant, a glucose sensor implant, a neurostimulator, a cochlear implant, an endoprostheses for closing persistent foramen ovale, an endoprostheses for closing an atrial septal defect, a left atrial appendage closure device, a pacemaker, a leadless pacemaker, a defibrillator, a prosthetic heart valve, preferably a TAVI/TAVR valve.

EE. Seamlessly joined or connected tissue or tissue component(s) according to embodiment 24 or obtained by a method according to any one of the embodiments A to T for medical use, in particular for use in a cardiovascular implant, a endovascular prostheses, an endoprostheses, an esophageal implants, a bile duct implant, a dental implant, an orthopedic implant, a sensory implant, a neurological implant a microchip containing implant.

FF. Seamlessly joined or connected tissue or tissue component(s) according to embodiment W or obtained by a method according to any one of the embodiments A to T for medical use, in particular for use in a stent, a vascular stent, a drug eluting stent, a pulmonary valve stent, a bile duct stent, a peripheral stent, a mitral stent, a stent graft, a venous valve, a tooth implant, a bone implant, a glucose sensor implant, a neurostimulator, a cochlear implant, an endoprostheses for closing persistent foramen ovale, an endoprostheses for closing an atrial septal defect, a left atrial appendage closure device, a pacemaker, a leadless pacemaker, a defibrillator, a prosthetic heart valve, preferably a TAVI/TAVR valve.

GG. Seamless connected tissue including a piece of tissue having at least two tissue parts overlapping each other and the at least two tissue parts overlapping each other are materially bonded to each other via crosslinked groups of the tissue.

HH. Seamless connected tissue according to claim GG, wherein the tissue is selected from biological tissue, autologous, xenogeneic, allogeneic tissue or collagen containing tissue.

II. Seamless connected tissue according to claim GG or HH, wherein the tissue is selected from pericardial tissue, connective tissue, peritoneal tissue, dura mater, tela submucosa, skin, ligament, tendons.

JJ. Seamless connected tissue according to any one of claims GG to II6, wherein the at least two tissue parts overlapping each other are materially bonded to each other via crosslinked amino groups of collagen fibers of the tissue, preferably being crosslinked by an aldehyde, preferably glutaraldehyde.

KK. Seamless connected tissue including at least one first piece of tissue and least one second piece of tissue, wherein at least one part of the at least one first piece of tissue overlaps with at least one part of the at least one second piece of tissue and the at least one part of the at least one first piece of tissue overlapping with the at least one part of the at least one second piece of tissue is materially bonded to each other via crosslinked groups of the at least one part of the at least one first piece of tissue and the at least one part of the at least one second piece of tissue

LL. Seamless connected tissue according to claim KK, wherein the at least one first piece of tissue and/or the at least one second piece of tissue is selected from biological tissue, autologous, xenogeneic, allogeneic tissue or collagen containing tissue.

MM. Seamless connected tissue according to claim KK or LL, wherein the at least one first piece of tissue and/or the at least one second piece of tissue is selected from pericardial tissue, connective tissue, peritoneal tissue, dura mater, tela submucosa, skin, ligament, tendons.

NN. Seamless connected tissue according to any one of claims KK to MM, wherein the at least one part of the at least one first piece of tissue overlapping with the at least one part of the at least one second piece of tissue are materially bonded to each other via crosslinked amino groups of collagen fibers of the at least one part of the at least one first piece of tissue and amino groups of collagen fibers of the at least one part of the at least one second piece of tissue, preferably being crosslinked by an aldehyde, preferably glutaraldehyde.

FIG. 1 shows a tissue placement of planar tissue patches/components porcine pericardium for a subsequent seamless joining/connection of two tissue patches porcine pericardium according to a process according to the invention. By tweezers 5, for example, a first rectangular joining partner 1—tissue patch of porcine pericardium—is placed on a suitable support surface 4 with a part forming the desired overlap area of the first joining partner 3, whereupon, by tweezers 5, for example, a second rectangular joining partner 4 is placed on the support surface 4. A second rectangular joining partner 2—tissue patch from porcine pericardium—is placed on the suitable support surface 4 in such a way that a part of the second joining partner 2 comes to lie overlapping with the first joining partner 1 in the desired overlap area 3. FIG. 1 thus represents an exemplary initial shape for the subsequent static, quasi-static or periodic pulsatile pressure loading/compression of porcine pericardial tissue components in the presence of a suitable crosslinking agent. The support surface 4 has two holes 4a, 4b for fixing and stacking the support surface(s).

FIG. 2 shows a planar seamlessly connected/joined porcine pericardium 1+2 with a crosslinked overlap region 6 after passing through a periodic pulsatile pressure loading/compression of porcine pericardial tissue components according to the invention in the presence of a suitable crosslinking agent; in this case glutaraldehyde solution. The joined pericardium rests on a support 4.

FIG. 3 shows tissue placement of a one-piece, complete tissue component of porcine pericardium 7 on a three-dimensional device for the valve component of an artificial aortic valve (TAVI/TAVR; 11, 12, 13) with negatives for three leaflets 8 and an inner skirt 9, which is used for subsequent three-dimensional crosslinking by static, quasi-static or periodic pulsatile pressure loading/compression, for example, in order to join the open tissue ends of the tissue component in the area 10 in a subsequent seamless joining/connecting according to one of the processes according to the invention. FIG. 3 thus represents, inter alia, an exemplary three-dimensional initial shape for the subsequent quasi-static or periodic pulsatile pressure loading/compression of porcine pericardial tissue components in the presence of a suitable crosslinking agent. The 12 is a holder of the device for clamping/fixing into a suitable crosslinking device.

FIG. 4 shows a one-piece, seamlessly connected/joined tissue component (here a one-piece valve component) with a leaflet portion 14 and an inner skirt 15, which is suitable for realizing a valve function in an artificial aortic valve arranged/fixed to a suitable support structure/stent. The valve component includes individually imprinted leaflets 16 as well as a continuous inner skirt 17, as well as recesses in the lower region for a precisely fitting insertion in an inlet region of a stent; 18 and 19.

FIG. 5 shows an exemplary construction of an inflatable sleeve 21 for the sutureless joining/connection of a TAVI/TAVR valve 20, which is suitable for radial static, quasi-static or periodic pulsatile pressure loading/compression. Shown is a stent framework 22 including a valve component 23 fixed into a pressure cylinder 24 of the inflatable cuff, wherein compressed air can be injected via a nozzle 25 with a channel 26. For example, to expand a balloon located in the center, which in turn exerts the pressure load, for example from the inside, on the valve component to be connected.

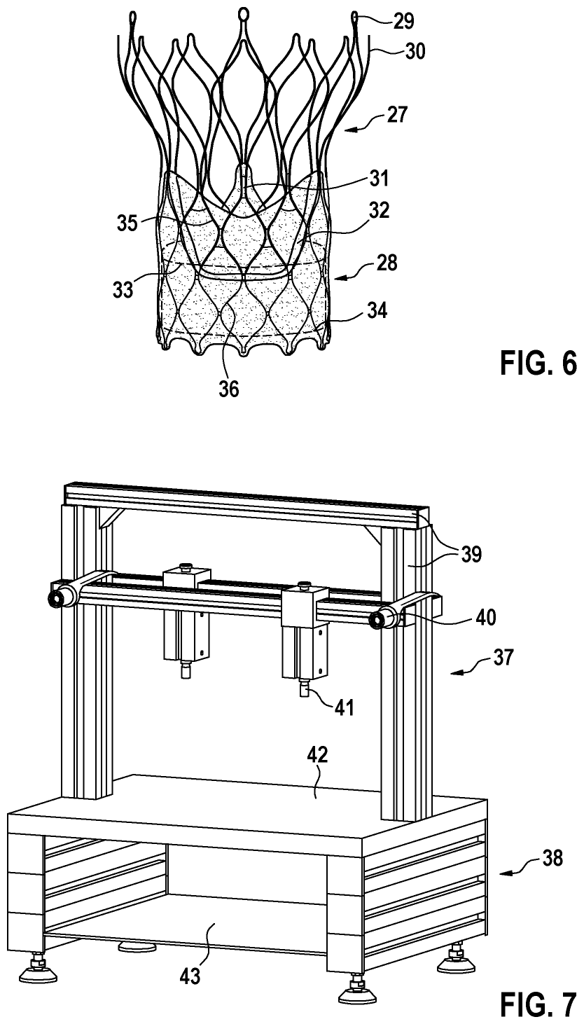

FIG. 6 shows an exemplary TAVI/TAVR valve with a self-expanding nitinol stent 27 having struts 35, a seamlessly joined valve component 28 according to the invention, in such a way that the stent component 27 is completely enclosed in the tissue of the valve component in the valve region 32 and has been completely enclosed by the static, quasi-static or periodic pulsatile pressure load according to the invention during chemical crosslinking with glutaraldehyde. Furthermore, in the lower region of the stent component (in the direction of influence), an inner skirt 33, 34 is shown as a dashed line in its contours. Thus, in this exemplary embodiment of a self-expanding TAVI/TAVR valve, as a technical effect, the surgical sutures typically required for the arrangement/fixation of the valve and skirt components could be significantly reduced, since both the one-piece valve component 28 as well as the one-piece inner skirt component 33, 34 have been connected/joined by the crosslinking technique according to the invention, and in particular the stent in the valve area has been inserted into the tissue used 36 and firmly enclosed via the seamless connections of the inner and outer sides of the double-layer tissue used here. If necessary, surgical sutures may still be required at the commissures of a stent 31 to suspend the valve component, or individual surgical sutures in the lower area of the stent inlet to additionally fix the respective one-piece valve or inner skirt component here. Furthermore, the exemplary TAVI/TAVR valve shown in FIG. 6 can additionally include an outer skirt component, which is also seamlessly connected/joined to the outer side of the tissue valve component via a process according to the invention. The outer skirt may have one or more three-dimensional protrusions, protrusions, or protrusions around its circumference, all of which are suitable for sealing against paravalvular leakage.

FIG. 7 shows an exemplary structure of a device 37 for applying static, quasi-static, or periodic pulsatile pressure to an overlap region of a tissue/component to be joined. The device includes a standing table 38 with a support surface 43 and a holder 39 with an adjustable rail system 40 for the suspension of two pneumatic cylinders 41 which can exert a static, quasi-static or periodic pulsatile pressure on an overlap area of a tissue on the table surface 42.

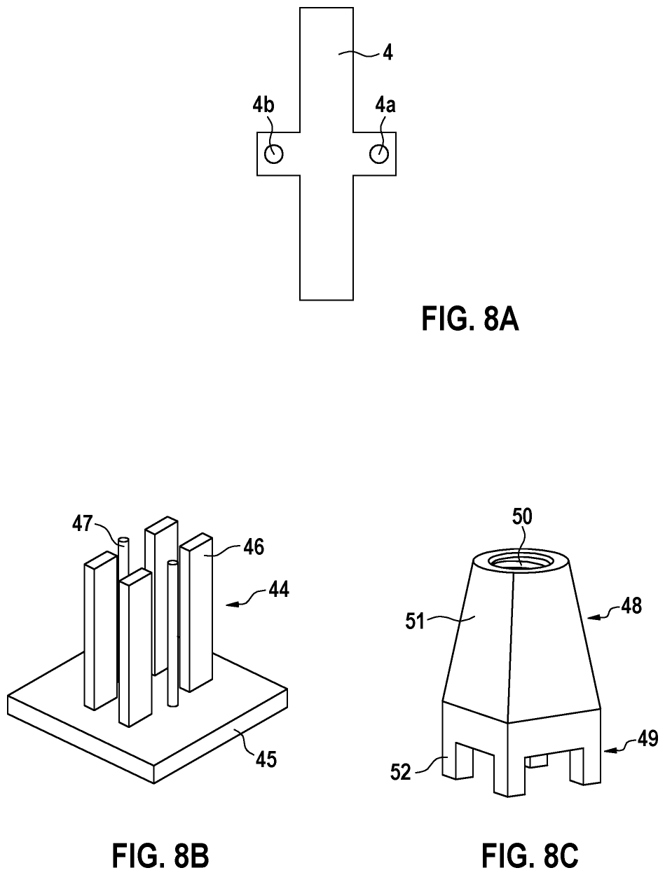

FIG. 8A shows an exemplary support surface 4 for tissue to be joined/connected with two holes 4a, 4b for fixing and stacking the support surface(s). FIG. 8B shows a receiving unit 44 for one or more of the support surfaces 4 according to FIG. 8A. The receiving unit 44 includes a base 45, four retaining webs 46 and two fixing rods 47 for the holes 4a, 4b of each support surface 4, which may be stacked one on top of the other. FIG. 8C shows a stamping device 48 which fits exactly to the support on the previously described support unit, and via its thread/holder 50 transfers the applied pressure load to the tissues with overlap areas enclosed in the support unit. The stamping device has pyramidal shaped walls 51 and several legs 49, 52.

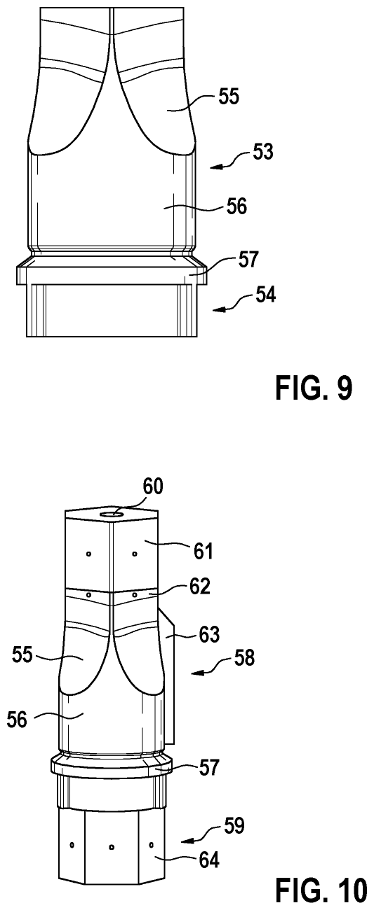

FIG. 9 shows a three-dimensional molded body 53 with a support surface seamless joining/connection of a one-piece valve component of a TAVI/TAVR valve with three negatives for leaflets 55, a cylindrical support surface for the inner skirt 56 and a holder 54 with a retaining ring 57 for clamping the molded body in a crosslinking unit for a process according to the invention.

FIG. 10 shows an assembled variant of the molded body described previously in FIG. 9 for clamping in a crosslinking unit via the additionally added edge elements 61, 62, 63, and 64 on the holder. The holder further includes at least one trough hole 60.

FIG. 11 shows a fully assembled variant 65 of multiple stacked support surfaces 4 with tissues to be joined/crosslinked in the receiving unit 44 of FIG. 8B and a stamping device 48 already pressurized according to FIG. 8C 48.

FIG. 12 shows a negative of a component of the molded body 53 according to FIG. 9 from a rear left perspective. With the negative for a leaflet 70 as well as a side wall 69 and two annular grooves 67, 68.

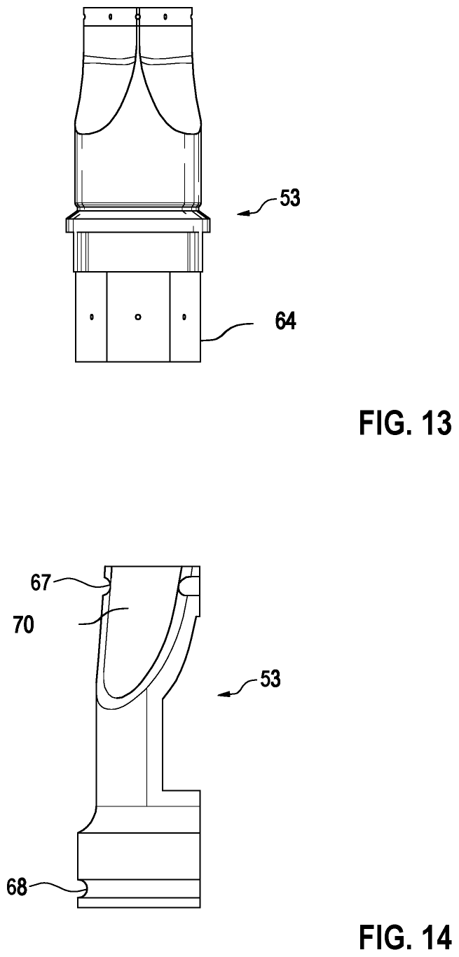

FIG. 13 again shows an assembled variant of the molded body 53 previously described in FIG. 9 for clamping in a crosslinking unit via the additionally added edge elements 64.

FIG. 14 shows a negative of a component of the molded body 53 according to FIG. 9 from a front right perspective. With the negative 70 for a leaflet as well as a part of the inner skirt and two annular grooves 67, 68.

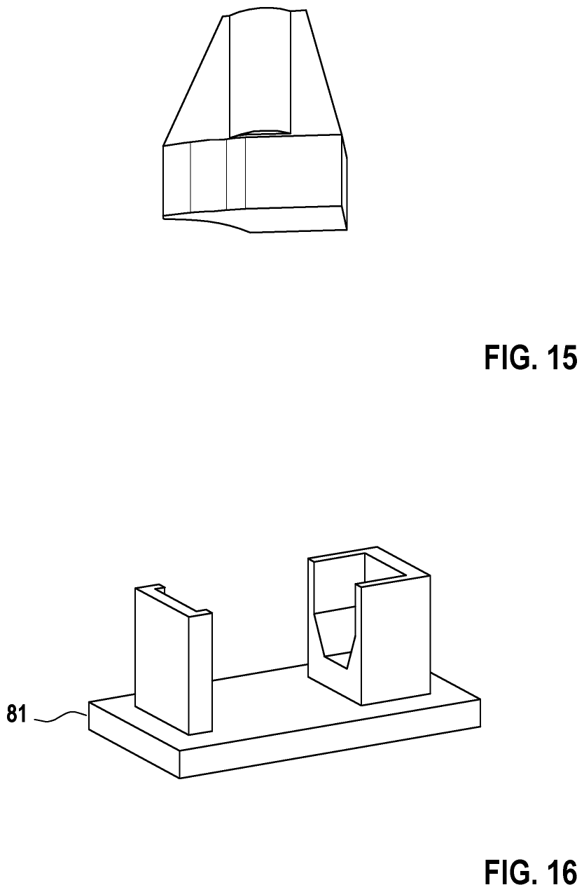

FIG. 15 shows another variant of a pressure die 80 which can be used, for example, to realize a seamless tissue connection of a one-piece TAVI/TAVR valve. See FIGS. 16, 17, 18.

FIG. 16 shows an exemplary holder 81 of a crosslinking unit for seamless joining of a one-piece tissue component of a TAVI/TAVR valve.

FIG. 17 shows a molded body 76 clamped for a TAVI/TAVR valve in the holder 81 of FIG. 16. The molded body 76 is clamped with an accurate fit with the grooves 72 and 73 between the holder jaws 74 and 75 with an overlying support surface 71 for forming the seamless connection/joint of the one-piece tissue component of a TAVI/TAVR valve.

FIG. 18 shows the device according to FIG. 17, but with pressure-loaded stamp element 78 on the support surface 71 of FIG. 17, whereby under suitable crosslinking conditions in the presence of a suitable crosslinking agent, a seamless joining of a one-piece valve component of a TAVI/TAVR valve can be realized.

1. A process for seamless, material bonded joining or connecting of a tissue or one or more tissue component(s), the process comprising:
providing one or more tissue(s) or tissue components comprising crosslinkable groups;
providing a suitable container, mold and/or support surface for the one or more tissue(s) or tissue component(s);
providing a device configured to receive the container, mold and/or support surface in a form-fit manner and configured to provide controllable static and/or quasi-static and/or periodic pulsatile and compression of the one or more tissue(s), or tissue component(s);
arranging one or more tissue(s) or tissue components(s) in the container, in the mold and/or on the support surface;
chemical crosslinking of overlap areas of the one or more tissue(s) or tissue component(s) under the action of the static, quasi-static or periodic pulsatile pressure compression, wherein the static pressure compression is applied via a force application in the range of 0.01 N/mm² to 10 N/mm² over a total period of at least 4 hours to 5 days or the quasi-static pressure compression is applied via a force application in the range of 0.01 N/mm² to 10 N/mm² over a time in the range of 200 seconds to 400 seconds in combination with a corresponding pressure release/pressure pause of 1 to 10 seconds over a total period of at least 4 hours to 5 days or the periodic pulsatile pressure compression is applied over a force input in the range of 0.01 N/mm² to 10 N/mm², over a time in the range of 10 seconds to 60 seconds in combination with a corresponding pressure release/pressure pause of 1 to 10 seconds over a total period of at least 4 hours to 5 days; and
removal of the one or more tissue(s) or tissue component(s) from the mold and/or the support surface.

2. The process according to claim 1, wherein
the device is configured to provide controllable static and/or quasi-static and/or periodic-pulsatile and substantially orthogonal compression of the one or more tissue(s) or tissue component(s), and wherein the device applies, during the chemical cross-linking, substantially orthogonal compression with a deviation of ±10° and in a range of 0.01 N/mm² to 10 N/mm².

3. The process according to claim 2, wherein:
the device applies 0.1 N/mm² to 1 N/mm², over a time in the range of 1 second to 15 minutes in combination with a corresponding pressure pause of 1 to 60 seconds, over a total period of at least 4 hours up to a maximum of 12 days.

4. The process according to claim 1, wherein the device applies static pressure compression during the chemical cross-linking in the range of 0.1 N/mm² to 1 N/mm², over a total period of at least 4 hours to 5 days.

5. The process according to claim 1, wherein the device applies quasi-static pressure compression during the chemical cross-linking in the range of 0.1 N/mm² to 1 N/mm², over a time in the range of 200 seconds to 400 seconds, in combination with a corresponding pressure pause of 1 to 10 seconds, over a total period of at least 4 hours to 5 days.

6. The process according to claim 1, wherein the device applies periodic pulsatile pressure compression in the range of 0.1 N/mm² to 1 N/mm² over a time in the range of 10 seconds to 60 seconds, in combination with a corresponding pressure pause of 1 to 10 seconds, over a total period of at least 4 hours to 5 days.

7. The process according to claim 1, wherein the device comprises an electronically controllable pneumatic cylinder, hydraulic cylinder or inflation sleeve.

8. The process according to claim 1, wherein the crosslinking agent is an aldehyde-containing solution or is selected from the group consisting of glutaraldehyde, carbodiimide, formaldehyde, glutaraldehyde acetals, acyl azides, cyanimide, genipin, tannin, pentagalloyl glucose, phytate, proanthocyanidin, reuterin or solutions thereof, and/or contains epoxy compounds.

9. The process according to claim 1, wherein the crosslinking agent is a 0.5% to 0.65% glutaraldehyde solution.

10. The process according to claim 1, wherein the one or more tissue(s)/tissue component(s) comprise chemically and/or biochemically crosslinkable groups.

11. (canceled)

12. (canceled)

13. (canceled)

14. (canceled)

15. The process according to claim 1, wherein the container, mold and/or support surface is configured to arranged and shape the one or more tissue(s) or tissue component(s) for the chemically cross-linking into a component for a medical implant selected from the group consisting of an artificial heart valve, in particular an artificial aortic valve, a coronary or peripheral vascular stent, a covered stent and/or a stent graft, a cardiovascular implant, an endovascular prostheses, an endoprostheses, a prosthetic heart valve, a TAVI/TAVR valve, an esophageal implant, a bile duct implant, a drug eluting stent, a pulmonary valve stent, a bile duct stent, a peripheral stent, a mitral stent, a stent graft, a venous valve, a dental implant, a bone implant, a cochlear implant, an endoprostheses for closing persistent foramen ovale, an endoprostheses for closing an atrial septal defect, a left atrial appendage closure device, a pacemaker, a leadless pacemaker or a defibrillator.

16. Seamless connected tissue comprising a piece of tissue having at least two tissue parts overlapping each other and the at least two tissue parts overlapping each other are materially bonded to each other via crosslinked groups of the tissue.

17. The seamless connected tissue according to claim 16, wherein the tissue is selected from biological tissue, autologous, xenogeneic, allogeneic tissue or collagen containing tissue.

18. The seamless connected tissue according to claim 16, wherein the tissue is selected from pericardial tissue, connective tissue, peritoneal tissue, dura mater, tela submucosa, skin, ligament, tendons.

19. The seamless connected tissue according to claim 16, wherein the at least two tissue parts overlapping each other are materially bonded to each other via crosslinked amino groups of collagen fibers of the tissue.

20. Seamless connected tissue comprising at least one first piece of tissue and least one second piece of tissue, wherein at least one part of the at least one first piece of tissue overlaps with at least one part of the at least one second piece of tissue and the at least one part of the at least one first piece of tissue overlapping with the at least one part of the at least one second piece of tissue are materially bonded to each other via crosslinked groups of the at least one part of the at least one first piece of tissue and the at least one part of the at least one second piece of tissue.

21. The seamless connected tissue according to claim 20, wherein the at least one first piece of tissue and/or the at least one second piece of tissue is selected from biological tissue, autologous, xenogeneic, allogeneic tissue or collagen containing tissue.

22. The seamless connected tissue according to claim 20, wherein the at least one first piece of tissue and/or the at least one second piece of tissue is selected from pericardial tissue, connective tissue, peritoneal tissue, dura mater, tela submucosa, skin, ligament, tendons.

23. The seamless connected tissue according to claim 20, wherein the at least one part of the at least one first piece of tissue overlapping with the at least one part of the at least one second piece of tissue are materially bonded to each other via crosslinked amino groups of collagen fibers of the at least one part of the at least one first piece of tissue and amino groups of collagen fibers of the at least one part of the at least one second piece of tissue.

24. The process according to claim 1, wherein the arranging arranges the one or more tissue component(s) to form one or more overlap areas.

25. The seamless connected tissue according to claim 16, wherein the crosslinked amino groups of collagen fibers are crosslinked by an aldehyde.

26. The seamless connected tissue according to claim 20, wherein the crosslinked amino groups of collagen fibers are crosslinked by an aldehyde.
